SEQUENCE LISTING (17 sequences)

SEQ ID NO: 1 - protein, 821 aa, artificial sequence
  S7 or pepL
MEMPGRRSNYTLLSQFSDDQVSVSVTGAPPPHYDSLSSENRSNHNSGNTGKAKAERGGFDWDPSGGGGGDHRLNNQPNRVGNNMYASSLGLQRQSSGSSFGESSLSGDYYMPTLSAAANEIESVGFPQDDGFRLGFGGGGGDLRIQMAADSAGGSSSGKSWAQQTEESYQLQLALALRLSSEATCADDPNFLDPVPDESALRTSPSSAETVSHRFWVNGCLSYYDKVPDGFYMMNGLDPYIWTLCIDLHESGRIPSIESLRAVDSGVDSSLEAIIVDRRSDPAFKELHNRVHDISCSCITTKEVVDQLAKLICNRMGGPVIMGEDELVPMWKECIDGLKEIFKVVVPIGSLSVGLCRHRALLFKVLADIIDLPCRIAKGCKYCNRDDAASCLVRFGLDREYLVDLVGKPGHLWEPDSLLNGPSSISISSPLRFPRPKPVEPAVDFRLLAKQYFSDSQSLNLVFDPASDDMGFSMFHRQYDNPGGENDALAENGGGSLPPSANMPPQNMMRASNQIEAAPMNAPPISQPVPNRANRELGLDGDDMDIPWCDLNIKEKIGAGSFGTVHRAEWHGSDVAVKILMEQDFHAERVNEFLREVAIMKRLRHPNIVLFMGAVTQPPNLSIVTEYLSRGSLYRLLHKSGAREQLDERRRLSMAYDVAKGMNYLHNRNPPIVHRDLKSPNLLVDKKYTVKVCDFGLSRLKASTFLSSKSAAGTPEWMAPEVLRDEPSNEKSDVYSFGVILWELATLQQPWGNLNPAQVVAAVGFKCKRLEIPRNLNPQVAAIIEGCWTNEPWKRPSFATIMDLLRPLIKSAVPPPNRSDL

SEQ ID NO: 2 - protein, 5 aa, Artificial sequence
  G4S linker
GGGGS

SEQ ID NO: 3 - protein, 7 aa, Artificial sequence
  Non-specific control peptide
EDLGREK

SEQ ID NO: 4 - protein, 7 aa, Artificial sequence
  rNLS oligopeptide
VKRKKKP

SEQ ID NO: 5 - protein, 444 aa, Artificial sequence
  mouse IL27 with linked subunits of IL27B (EBI3) and IL27A (IL27p28)
MSKLLFLSLALWASRSPGYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPVPGVGVPGVGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 6 - protein, 454 aa, artificial sequence
  human IL27 linked subunits IL27B (EBI3) and IL27A (IL27p28)
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYIQVAAQDLTDYGELSDWSLPATATMSLGKVPGVGVPGVGFPRPPGRPQLSLQELRREFTVSLHLARKLLAEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 7 - protein, 455 aa, Artificial sequence
  CANINE IL27 with linked subunits of IL27B (EBI3) and IL27A (IL27p28)
MAPGLLLVLALWVGCSPCRGREGAPAAPTQPRVRCRASRYPVAVDCFWTLPPAPRSATPTSFIATYRLGVAAHGESLPCLQQTPEATSCTIPDVHMFSMVPYVLNVTAVRPWGSSSSFVPFVPEQLIKPDPPEGVRLSVLPRQRLWVQWEPPRSWPFPELFSLKYWIRYKHHGSPRFRQVGPIEATSFTFRAVRPQARYCIQVAAQDLTDYGESSDWSLPAAPSTPLGKVPGVGVPGVGFPRPPGRSPLSLQELRREFKVSLQLAKKLFSEVRIQAHHFAESQLPGVSLDLLPLGDQLPNVSLPFQAWHSLSDPERLCFLSMMLHPFHALLESLGSQGGWTSSEKMHLWTMRLDLRDLQRHLRFQVEYPPTCSTPRDQQEEEEEQHEERKGLLAAAPGGPSQTAVQPSWPQLLYTYQLLHSLELALARAVRDLLLLSQAGNPAPPVGHSTFGSQP

SEQ ID NO: 8 - protein, 12 aa, Artificial sequence
  polypeptide targeting EG
YHWYGYTPQNVI

SEQ ID NO: 9 - protein, 7 aa, Artificial sequence
  peptide targeting GRP78
SNTRVAP

SEQ ID NO: 10 - protein, 15 aa, Artificial sequence
  peptide targeting BMPR1b
AISMLYLDENEKVVL

SEQ ID NO: 11 - protein, 12 aa, Artificial sequence
  targeting petpide 11
TPLSYLKGLVTV

SEQ ID NO: 12 - protein, 12 aa, Artificial sequence
  targeting peptide 12
NPYHPTIPQSVH

SEQ ID NO: 13 - protein, 7 aa, Artificial sequence
  targeting peptide 13
ASACPPH

SEQ ID NO: 14 - protein, 9 aa, Artificial sequence
  Targeting peptide 14
GGPNLTGRW

SEQ ID NO: 15 - protein, 7 aa, Artificial sequence
  targeting petpide 15
FLPASGL

SEQ ID NO: 16 - protein, 8 aa, Artificial sequence
  targeting petpide 16
TPIVHHVA

SEQ ID NO: 17 - protein, 11 aa, Artificial Sequence
  ACE2p targeting peptide
TVALPGGYVRV

(19) United States
(12) Patent Application Publication
(10) Pub. No.: US 2023/0111460 A1
(43) Pub. Date: Apr. 13, 2023

(54) LIGAND-MEDIATED DELIVERY OF THERAPEUTIC PROTEINS AND THE USES THEREOF

(71) Applicant: Purdue Research Foundation, West Lafayette, IN (US)

(72) Inventor: Marxa L. Figueiredo, Lafayette, IN (US)

(73) Assignee: Purdue Research Foundation, West Lafayette, IN (US)

(21) Appl. No.: 17/794,917

(22) PCT Filed: Jan. 1, 2021
(86) PCT No.: PCT/US2021/012003

§ 371 Date: Jul. 22, 2022

Related U.S. Application Data
(60) Provisional application No. 62/967,767, filed on Jan. 30, 2020.

(51) Int. Cl. C07K 14/54; A61K 47/64; A61K 41/00; A61K 48/00; A61P 35/00

(52) U.S. Cl. CPC C07K 14/54; A61K 47/64; A61K 41/0047; A61K 48/0083; A61K 48/0041; A61K 47/6455; A61P 35/00; C07K 2319/09

(57) ABSTRACT

The present invention generally relates to composition matters and methods useful for gene delivery and an option for therapeutic treatment of various diseases. Particularly, this disclosure relates to a plasmid vector comprising a fusion of a plurality of genes comprising a gene of a chemokine or a cytokine, a gene for a targeting polypeptide and genes for one or more polypeptide linkers. Methods of use and composition matters are within the scope of this disclosure.

### CROSS-REFERENCE TO RELATED APPLICATIONS

The present PCT patent application relates to and claims the priority benefit of U.S. Provisional Patent Application Ser. No. 62/967,767, filed Jan. 30, 2020, the content of which is hereby incorporated by reference in its entirety.

### STATEMENT OF SEQUENCE LISTING

A computer-readable form (CRF) of the Sequence Listing is submitted with this application. The file, entitled 68875-02_Seq_Listing_ST25_txt, is generated on Dec. 28, 2020. Applicant states that the content of the computer-readable form is the same and the information recorded in computer readable form is identical to the written sequence listing herein.

### GOVERNMENT SUPPORTING CLAUSE

This invention was made with government support under contracts CA196947 and AR069079, both awarded by the National Institutes of Health. The government has certain rights in the invention.

### TECHNICAL FIELD

The present invention generally relates to composition matter and methods useful for gene delivery and an option for therapeutic treatment of various diseases, in particular, to a plasmid vector comprising a fusion of a plurality of genes of chemokine or cytokine, a targeting polypeptide together with one or more linkers. Methods of use and composition matters are within the scope of this disclosure.

### BACKGROUND AND BRIEF SUMMARY OF INVENTIONS

This section introduces aspects that may help facilitate a better understanding of the disclosure. Accordingly, these statements are to be read in this light and are not to be understood as admissions about what is or is not prior art.

Our group and others have previously shown cytokine Interleukin-27 (IL-27) to be a promising therapeutic for arthritis¹ and malignant tumors²⁻⁴, based on its multifunctional (immune stimulatory, anti-angiogenic, pro-osteogenic) activity. For example, IL-27 helped prevent osteoclast formation and promote osteoblast differentiation^{2,3}, key therapeutic features for treating bone-metastatic tumors. As such, in vivo gene delivery of IL-27 significantly reduced the rate of tumor growth and normalized bone density⁴. IL-27 is a heterodimeric cytokine composed of subunits IL-27p28 and EBI3 (Epstein-Barr virus-induced gene 3), which are related to the IL-12 subunits p35 and p40, respectively. IL-27 is immunomodulatory and was originally thought to be produced mainly by antigen-presenting cells in response to microbial or host immune stimuli. However, IL-27 recently has been shown to be involved in regulating immune response against tumor development and in serving as an ‘alarm’ to sense inflammatory or infectious response to promote bone repair⁵. The receptor for IL-27, a heterodimer composed of WSX1 and gp130 subunits, is highly expressed in lymphoid organs, bone, normal and tumor epithelial cells^{6, 7}, melanoma^{s}, and leukemia⁹. IL-27 signaling induces T-bet, IFNγ, and IL12-Rβ2 expression, promoting initiation of Thl differentiation^{10,11}. Either systemic¹² or intratumoral² IL-27 treatments eliminate tumors without toxicity. IL-27 also shows antitumor activity through indirect mechanisms such as induction of natural killer and cytotoxic T lymphocyte responses or inhibition of angiogenesis through induction of CXCL9-10¹².

Regarding IL-27 therapy delivery in vivo, we selected a method that utilizes clinically safe ultrasound (US) frequencies to induce cellular cavitation and deliver plasmid DNA via sonoporation (i.e., sonodelivery)². Previous studies using this method showed that the gene delivery efficiency can approximate that of adenovirus². We have previously optimized sonodelivery conditions using reporter gene plasmids, finding that the best approach consisted of complexing plasmid DNA (pDNA) with a novel cationic polymer, termed rNLSd, in the presence of microbubble-assisted sonoporation¹³. In previous studies, we observed that wild-type IL-27 sonodelivery slowed bone destruction and inhibited tumor growth⁴. However, one limitation of that approach was its moderate efficacy, in which tumor growth rate was reduced but tumors were not completely eradicated. Very recently, IL-27 delivery has employed creative methods including incorporating the cytokine within peptide-conjugated liposomes (ART1-IL-27) for controlling autoimmune arthritis14. These ART IL-27 liposomes, when intravenously injected in arthritic rats, were more effective in suppressing disease progression than control-IL-27 liposomes lacking ART-1 or free IL-27 at an equivalent dose. ART-1-directed liposomal IL-27 offered a higher safety profile and an improved therapeutic index, supporting the concept that peptides can be used to target proteins or nanoparticles for targeted delivery including biologics or small molecule compounds with enhanced efficacy and reduced systemic exposure. We hypothesized that targeting the cytokine to tumor tissue by utilizing peptides that could bind receptors upregulated in tumor cells, such as the Interleukin-6 (IL-6) receptor, could help augment IL-27 bioactivity.

For the purpose of targeting the IL-6 receptor, we selected a candidate heptapeptide from the literature, LSLITRL (S7 or ‘pepL’; SEQ ID NO: 1), which was first identified from a 7-mer random cyclic phage display screen targeting the IL-6 receptor alpha subunit (IL-6Rα)¹⁵. This pepL inhibited IL-6 binding to IL-6Rα in a dose-dependent manner and could bind to the plasma membrane of IL-6Ra-expressing cell lines. The activity of pepL was attributed to its ability to antagonize IL-6 binding to IL-6Rα and inhibit phosphorylation of Akt and ERK1/2 MAPK. This peptide reduced in vivo C33A human cervical carcinoma growth by ˜75%, and induced apoptotic cell death in tumors, establishing pepL both as a therapeutic and a targeting peptide.

We have also reported the strategy of a “model” for cytokine engineering that would promote targeting by using —7-12 amino acid peptide ligands attached to the C-terminus of a cytokine via a short linker (GGGGS; SEQ ID NO: 2)¹⁶. This C-terminal modification of secreted molecules enables their targeting and accumulation at tumor sites. We examined this concept with a secreted luciferase (Gaussia Luc or GLuc) to mimic therapeutic cytokine secretion, targeting, and accumulation in tumors. Sonodelivery was employed with a biocompatible polymer complexed to pDNA to create a nanoplex, which was delivered along with microbubbles and sonicated to achieve ultrasound-enhanced muscle transfection.

There remains a lack of therapeutics that can simultaneously and effectively treat the prostate tumor while restoring affected bone tissue. Cytokine immunotherapies hold great promise because they are secreted molecules that can reach and treat both primary and distant secondary tumors. Thus, IL-27 targeting with a dual therapeutic and targeting C-terminal peptide, pepL, may augment cytokine bioactivity and efficacy against prostate tumors in vivo.

### BRIEF DESCRIPTION OF THE DRAWINGS

Embodiments of the present disclosure will now be described by way of example in greater detail with reference to the attached Figures, in which:

FIGS. 1A-1D depict that a C-term ‘peptide L’ (pepL) can target an engineered cytokine model protein (Gaussia Luc) to tumor cells. FIG. 1A shows alignment of mouse and human IL6-Rα illustrates the degree of structural homology between these two species; FIG. 1B shows that a model of pepL interactions with the mouse or human IL6Rα, as detailed in Materials and Methods. FIG. 1C demonstrates that STAT1- or STAT3-luc reporter assays show upregulation of STAT1 but also upregulation of STAT3 by the free pepL (a peptide targeting the IL6-Ra) relative to a non-specific control free peptide (ns pep). The engineering of the pepL or nonspecific control to an irrelevant protein (Gaussia Luc or Gluc) enabled pepL to activate STAT1 but not STAT3, relative to ns pep control. Cells were transfected with STAT3-luc reporter vector and treated with conditioned media (generated in C2C12 cells) containing either control or peptide-modified Gluc, as described in Materials and Methods. *, p<0.05 relative to control (ns pep or Gluc.ns) levels of STAT1- or STAT3-Luc activity. FIG. 1D shows an in vitro assay for detecting Gluc binding to cells. Gluc engineered at the C-term (Gluc-ns or pepL) were expressed from a mammalian expression vector in C2C12 muscle cells. The culture conditioned media (CCM) was collected and used in a binding assay using normal (AD293, HEPG2, or NHPre1), tumor cells (PC3, RM1, TC2R), or differentiating bone cells (OB, MC3T3E1-14 preosteoblasts and OC, RAW264.7 at day 4). *, p<0.05 compared to Gluc-ns CCM.

FIGS. 2A-2B demonstrate the sonodelivery of GLuc fusion proteins in vivo. FIG. 2A shows a schematic of sonodelivery for expressing Gaussia luciferase (GLuc) proteins in mouse muscle. A nanoplex is formed by rNLSd polymer, prepared as described in reference¹¹, complexed with plasmid DNA encoding GLuc. This nanoplex is delivered in the presence of microbubbles (MB) as described in Materials and Methods. An ultrasound stimulus (US) is applied to disrupt the MB and the nanoplex of polymer:pGluc mediates skeletal muscle cell transfection. The proteins secreted contain a C-terminal peptide tag that either targets the IL6-Rα (pepL) or is untargeted (non-specific peptide control). FIG. 2B shows an Ex vivo GLuc imaging post-gene delivery. Bioluminescence imaging is shown using coelenterazine substrate on organs isolated from animals receiving control (Gluc-ns) or ligand targeted GLuc (Gluc-pepL). color bar, p/sec/cm²/sr. Signals are present in the tumor:bone region only when targeted Gluc-pepL is delivered to muscle. Right plot, average Gluc signals from tumor:bone ex vivo pooled from day 10 and 14 post-delivery (*,p<0.013 for comparing GL.pepL relative to GL.ns accumulation in tumor tissue (accumulation in normal organs not significantly different between GL.ns and GL.pepL). Mice were bearing TC2R tumors intratibially.

FIGS. 3A-3C demonstrate that a ligand-targeted Interleukin-27 has enhanced bioactivity in vivo, stimulating STAT1 and IFNγ signaling in target cells. FIG. 3A shows a model of IL-27pepL showing IL-27p28 and EBI3 subunits, the G4S linker, and the pepL peptide; FIG. 3B shows the bioactivity of IL-27pepL in vivo using TC2Ras prostate cancer cells. Cells were transfected with luciferase reporter vectors containing either STAT1 binding sites or the IFNγ promoter to generate ‘reporter cells’. Equal numbers of reporter cells (7.7×10⁵) were implanted in the flanks of C57BL6 males (n=6) that had received in the hind thigh 3 days prior by sonoporation 12.5 μg of plasmid DNA (either empty control pMCS, IL-27 with a non-specific peptide (ns) at the C-terminus, or C-term-targeted IL-27 (IL-27pepL). pDNA were delivered via sonodelivery (polymer NLSd+ultrasound+MB). 24h post-cell injection (i.e. day 4 post-sonoporation of pDNA), the effect of IL-27ns or IL-27pepL can be visualized in the presence of luciferin substrate. Bioluminescent signals were detectable using an IVIS100 Xenogen imager only in animals that received pIL-27ns or pIL-27pepL but not pMCS control vector. Color bar, p/sec/cm²/sr. FIG. 3C shows the fold increase of Luciferase activity of pIL-27ns or pIL-27pepL compared to pMCS-treated. Animals treated with pIL-27ns had an increase of Luc activity compared to pMCS control vector (*, p<0.04). The animals receiving pIL-27pepL had a further increase in Luc activity relative to the pIL-27ns treated sites (#, p<0.03).

FIGS. 4A-4B demonstrate the targeted IL-27 utilizes both paracrine and autocrine signaling. FIG. 4A shows pepL-modified IL-27 utilizes autocrine mode of signaling. In the Autocrine design, the plasmid expressing IL-27 was delivered along with the reporter plasmid (STAT1/GAS/ISRE-Luc or STAT1-luc). The IL-27 C-term pepL (IL-27pepL) allows anchoring of cytokine to the overexpressed targeting receptors (IL6Rα). The cytokine is expressed and acts on the IL27R to mediate STAT1 signaling. FIG. 4B shows the PepL enhances IL-27 signaling also in a paracrine mode. In the paracrine design, either differentiating osteoblast (OB, MC3T3E1-14 day 4) or epithelial cells (TC2r) were transfected with STAT1/GAS/ISRE-Luc (STAT1-luc), then mixed with the other cell type expressing IL-27ns, IL-27pepL or empty vector ctrl. In order to signal, IL-27pepL had to be secreted from one cell type and bind to the other cell type (bearing STAT1-luc) to induce signaling. In the autocrine design, pSTAT1-Luc and pIL-27s were cotransfected. The paracrine signaling effect can be blocked by pretreatment (30 min) with an anti-IL6Ra blocking antibody (Ab). *, p<0.04 vs ctrl, #, p<0.05 vs IL-27ns. *, p<0.05 vs ctrl mcs or no cell coculture (comix); #, p<0.05 vs 27ns; $, p<0.05 AB 27L vs 27L

FIGS. 5A-5D demonstrate the differential gene expression by qPCR analysis following gene delivery in TC2R. Following gene delivery of TC2R cells with either control (pMCS), pIL27ns, or pIL27pepL, and qPCR analysis, the cells transfected with pIL27ns or pIL27pepL had different patterns of up-(red) and down-regulation (blue) of gene expression relative to control. Fold changes in expression relative to control pMCS are shown at 24h-post transfection in: FIG. 5A shows the genes delivered (IL27p28 and EBI3), FIG. 5B shows the IL-6 and IL-27 responsive or target genes, FIG. 5C shows the genes representing cytokines in the tumor microenvironment, and FIG. 5D shows the immunogenic genes. *, p<0.05 relative to control pMCS transfected cells; #, p<0.05 relative to pIL27.ns transfected cells.

FIGS. 6A-6B depict a Heatmap of canonical pathways predicted by IPA to be altered between cells expressing IL27ns and IL27pepL. A comparison analysis was performed between samples of TC2R cells transfected with plasmid expressing IL27ns and IL27pepL (both corrected to pMCS vector control) as per the IPA analyses described in Materials and Methods. FIG. 6A shows the Canonical pathways that differ between the IL27.ns and IL27.pepL treatments. Color bar, activation z-scores; and FIG. 6B shows the Cellular and Organismal Functions that differ between the IL-27ns and IL-27pepL treatments. Color bar, −log(B-H p-value).

FIGS. 7A-7C demonstrate that IL-27 targeting enhances antitumor activity in vivo. FIG.

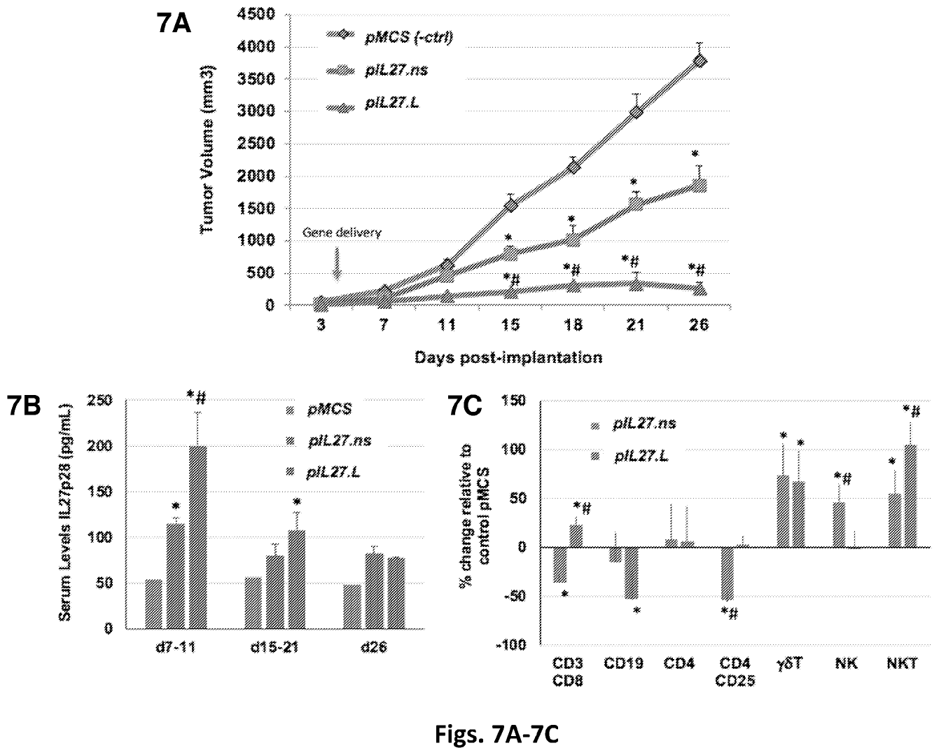

7A shows a TC2R prostate tumor model. Cancer cells were subcutaneously implanted in C57/BL6 male mice and tumor growth followed by caliper measurements over time and is expressed in mm³. pIL-27-pepL is more effective than pIL-27ns and an empty vector control (pMCS) in reducing TC2R tumor growth. Plasmids (12.5 μg) encoding pMCS, pIL-27ns, or pIL-27pepL were delivered by I.M. sonoporation to the hind thigh complexed to NLSd polymer in the presence of microbubbles and ultrasound as described in Materials and Methods. *, p<0.05 compared to pMCS-treated control tumors; #, p<0.05 compared to mice treated with pIL-27ns. FIG. 7B shows the serum levels of IL-27 were not significantly different among animals receiving pIL-27ns or pIL-27pepL in general, except for the early timepoints (day 7-11) (*, p<0.05). FIG. 7C demonstrates that IL-27 targeting enhances effector cell recruitment to TC2R prostate tumors.*, p<0.05 compared to pMCS; #, p<0.05 compared to pIL27ns.

Table 1. qPCR data analyzed by Ingenuity Pathway Analysis—Upstream regulators per treatment—predicted activation or inhibition and their target molecules in the dataset.

### BRIEF DESCRIPTION OF SEQUENCE LISTING

SEQ ID NOs: 1 and 8-17 are targeting polypeptides:

(SEQ ID NO: 1)
Leu-Ser-Leu-Ile-Thr-Arg-Leu;
(SEQ ID NO: 8)
YHWYGYTPQNVI;
(SEQ ID NO: 9)
SNTRVAP;
(SEQ ID NO: 10)
AISMLYLDENEKVVL;
(SEQ ID NO: 11)
TPLSYLKGLVTV;
(SEQ ID NO: 12)
NPYHPTIPQSVH;
(SEQ ID NO: 13)
ASACPPH;
(SEQ ID NO: 14)
GGPNLTGRW;
(SEQ ID NO: 15)
FLPASGL,
(SEQ ID NO: 16)
TPIVHHVA,
and
(SEQ ID NO: 17)
TVALPGGYVRV.

SEQ ID NO: 2, Gly-Gly-Gly-Gly-Ser is a linker peptide.

SEQ ID NO: 3, EDLGREK is a non-specific control peptide.

SEQ ID NO: 4, Val-Lys-Arg-Lys-Lys-Lys-Pro is a pendant peptide for the polymer used in the formulation.

IL-27 with linked subunits IL27B (EBI3) and IL27A (IL27p28) of mouse:

(SEQ ID NO: 5)
MSKLLFLSLALWASRSPGYTETALVALSQPRVQCH
ASRYPVAVDCSWTPLQAPNSTRSTSFIATYRLGVA
TQQQSQPCLQRSPQASRCTIPDVHLFSTVPYMLNV
TAVHPGGASSSLLAFVAERIIKPDPPEGVRLRTAG
QRLQVLWHPPASWPFPDIFSLKYRLRYRRRGASHF
RQVGPIEATTFTLRNSKPHAKYCIQVSAQDLTDYG
KPSDWSLPGQVESAPHKPVPGVGVPGVGFPTDPLS
LQELRREFTVSLYLARKLLSEVQGYVHSFAESRLP
GVNLDLLPLGYHLPNVSLTFQAWHHLSDSERLCFL
ATTLRPFPAMLGGLGTQGTWTSSEREQLWAMRLDL
RDLHRHLRFQVLAAGFKCSKEEEDKEEEEEEEEEE
KKLPLGALGGPNQVSSQVSWPQLLYTYQLLHSLEL
VLSRAVRDLLLLSLPRRPGSAWDS.
CXCL9

For human IL27 linked subunits IL27B (EBI3) and IL27A (IL27p28):

(SEQ ID NO: 6)
MTPQLLLALVLWASCPPCSGRKGPPAALTLPRVQC
RASRYPIAVDCSWTLPPAPNSTSPVSFIATYRLGM
AARGHSWPCLQQTPTSTSCTITDVQLFSMAPYVLN
VTAVHPWGSSSSFVPFITEHIIKPDPPEGVRLSPL
AERQLQVQWEPPGSWPFPEIFSLKYWIRYKRQGAA
RFHRVGPIEATSFILRAVRPRARYYIQVAAQDLTD
YGELSDWSLPATATMSLGKVPGVGVPGVGFPRPPG
RPQLSLQELRREFTVSLHLARKLLAEVRGQAHRFA
ESHLPGVNLYLLPLGEQLPDVSLTFQAWRRLSDPE
RLCFISTTLQPFHALLGGLGTQGRWTNMERMQLWA
MRLDLRDLQRHLRFQVLAAGFNLPEEEEEEEEEEE
EERKGLLPGALGSALQGPAQVSWPQLLSTYRLLHS
LELVLSRAVRELLLLSKAGHSVWPLGFPTLSPQP.

For canine IL27 linked subunits IL27B (EBI3) and IL27A (IL27p28):

(SEQ ID NO: 7)
MAPGLLLVLALWVGCSPCRGREGAPAAPTQPRVRC
RASRYPVAVDCFWTLPPAPRSATPTSFIATYRLGV
AAHGESLPCLQQTPEATSCTIPDVHMFSMVPYVLN
VTAVRPWGSSSSFVPFVPEQLIKPDPPEGVRLSVL
PRQRLWVQWEPPRSWPFPELFSLKYWIRYKHHGSP
RFRQVGPIEATSFTFRAVRPQARYCIQVAAQDLTD
YGESSDWSLPAAPSTPLGKVPGVGVPGVGFPRPPG
RSPLSLQELRREFKVSLQLAKKLFSEVRIQAHHFA
ESQLPGVSLDLLPLGDQLPNVSLPFQAWHSLSDPE
RLCFLSMMLHPFHALLESLGSQGGWTSSEKMHLWT
MRLDLRDLQRHLRFQVEYPPTCSTPRDQQEEEEEQ
HEERKGLLAAAPGGPSQTAVQPSWPQLLYTYQLLH
SLELALARAVRDLLLLSQAGNPAPPVGHSTFGSQP.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 20%, within 10%, within 5%, or within 1% of a stated value or of a stated limit of a range.

In the present disclosure the term “substantial” or “substantially” can allow for a degree of variability in a value or range, for example, within 80%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, the disclosure of which is hereby incorporated by reference.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” or “subject” includes a human and non-human animals such as companion animals (dogs and cats and the like) and livestock animals. Livestock animals are animals raised for food production. The patient to be treated is preferably a mammal, in particular a human being.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides.

The terms “polypeptide,” “peptide,” and “protein” are used interchangeably herein (unless expressly stated otherwise) to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it is positioned so as to facilitate translation. Generally, “operably linked” means that the DNA sequences being linked are contiguous and, in the case of leader, contiguous and in a reading phase. However, enhancers do not necessarily have to be contiguous. Linking may be accomplished by ligation at convenient restriction sites. If such sites do not exist, synthetic oligonucleotide adaptors or linkers may be used in accordance with conventional practice.

“Percent (%) amino acid sequence identity” with respect to a reference to a polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity and not considering any conservative substitutions as part of the sequence identity. Alignment for purposes of determining percent amino acid sequence identity can be achieve din various ways that are within the skill of the art, for instance, using publicly available computer software. Those skilled in the art can determine appropriate parameters for aligning sequences, including any algorithms needed to achieve maximal alignment over the full length of the sequences being compared.

The terms “treatment” or “therapy” as used herein (and grammatical variations thereof such as “treat, “treating,” and “therapeutic”) include curative and/or prophylactic interventions in an attempt to alter the natural course of the individual being treated. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions of the present disclosure are used to delay development of a disease and/or tumor, or to slow (or even halt) the progression of a disease and/or tumor growth.

In some aspects, this invention generally relates to composition matter and methods useful for gene delivery and an option for therapeutic treatment of various diseases, in particular, to a plasmid vector comprising a fusion of a plurality of genes comprising that of a gene of chemokine or cytokine, a targeting polypeptide and one or more linkers. Methods of use and composition matters are within the scope of this disclosure.

In some illustrative embodiments, this disclosure relates to a composition matter comprising an engineered plasmid vector, wherein said vector comprises a fusion of a plurality of genes of a therapeutic chemokine or a cytokine, a targeting polypeptide, and one or more optional linkers.

In some illustrative embodiments, this disclosure relates to a composition matter comprising an engineered plasmid vector as disclosed herein, wherein said cytokine is selected from the group consisting of interleukin-27 (IL-27), IL27p28 (IL-30), Epstein-Barr virus-induced gene 3 (EBI3), IL-23, IL-18, IL-17, and any combination thereof.

In some illustrative embodiments, this disclosure relates to a composition matter comprising an engineered plasmid vector as disclosed herein, wherein said cytokine is origin of a mouse, a human, or a canine.

In some illustrative embodiments, this disclosure relates to a composition matter comprising an engineered plasmid vector as disclosed herein, wherein said cytokine is a IL-27 comprised of linked subunits of IL27B (EBI3) and IL27A (IL27p28) having a sequence of:

MSKLLFLSLALWASRSPGYTETALVALSQPRVQCH
ASRYPVAVDCSWTPLQAPNSTRSTSFIATYRLGVA
TQQQSQPCLQRSPQASRCTIPDVHLFSTVPYMLNV
TAVHPGGASSSLLAFVAERIIKPDPPEGVRLRTAG
QRLQVLWHPPASWPFPDIFSLKYRLRYRRRGASHF
RQVGPIEATTFTLRNSKPHAKYCIQVSAQDLTDYG
KPSDWSLPGQVESAPHKPVPGVGVPGVGFPTDPLS
LQELRREFTVSLYLARKLLSEVQGYVHSFAESRLP
GVNLDLLPLGYHLPNVSLTFQAWHHLSDSERLCFL
ATTLRPFPAMLGGLGTQGTWTSSEREQLWAMRLDL
RDLHRHLRFQVLAAGFKCSKEEEDKEEEEEEEEEE
KKLPLGALGGPNQVSSQVSWPQLLYTYQLLHSLEL
VLSRAVRDLLLLSLPRRPGSAWDS
(SEQ ID NO: 5; mouse IL27 with
linked subunits of IL27B (EBI3)
and IL27A (IL27p28));

or

MTPQLLLALVLWASCPPCSGRKGPPAALTLPRVQC
RASRYPIAVDCSWTLPPAPNSTSPVSFIATYRLGM
AARGHSWPCLQQTPTSTSCTITDVQLFSMAPYVLN
VTAVHPWGSSSSFVPFITEHIIKPDPPEGVRLSPL
AERQLQVQWEPPGSWPFPEIFSLKYWIRYKRQGAA
RFHRVGPIEATSFILRAVRPRARYYIQVAAQDLTD
YGELSDWSLPATATMSLGKVPGVGVPGVGFPRPPG
RPQLSLQELRREFTVSLHLARKLLAEVRGQAHRFA
ESHLPGVNLYLLPLGEQLPDVSLTFQAWRRLSDPE
RLCFISTTLQPFHALLGGLGTQGRWTNMERMQLWA
MRLDLRDLQRHLRFQVLAAGFNLPEEEEEEEEEEE
EERKGLLPGALGSALQGPAQVSWPQLLSTYRLLHS
LELVLSRAVRELLLLSKAGHSVWPLGFPTLSPQP

(SEQ ID NO: 6; human IL27 linked subunits IL27B (EBI3) and IL27A (IL27p28)) ; or

MAPGLLLVLALWVGCSPCRGREGAPAAPTQPRVRC
RASRYPVAVDCFWTLPPAPRSATPTSFIATYRLGV
AAHGESLPCLQQTPEATSCTIPDVHMFSMVPYVLN
VTAVRPWGSSSSFVPFVPEQLIKPDPPEGVRLSVL
PRQRLWVQWEPPRSWPFPELFSLKYWIRYKHHGSP
RFRQVGPIEATSFTFRAVRPQARYCIQVAAQDLTD
YGESSDWSLPAAPSTPLGKVPGVGVPGVGFPRPPG
RSPLSLQELRREFKVSLQLAKKLFSEVRIQAHHFA
ESQLPGVSLDLLPLGDQLPNVSLPFQAWHSLSDPE
RLCFLSMMLHPFHALLESLGSQGGWTSSEKMHLWT
MRLDLRDLQRHLRFQVEYPPTCSTPRDQQEEEEEQ
HEERKGLLAAAPGGPSQTAVQPSWPQLLYTYQLLH
SLELALARAVRDLLLLSQAGNPAPPVGHSTFGSQP

(SEQ ID NO: 7; CANINE IL27 with linked subunits of IL27B (EBI3) and IL27A (IL27p28)).

In some illustrative embodiments, this disclosure relates to a composition matter comprising an engineered plasmid vector as disclosed herein, wherein said targeting polypeptide further has therapeutic functions.

In some other illustrative embodiments, this disclosure relates to a composition matter comprising an engineered plasmid vector as disclosed herein, wherein said targeting polypeptide comprises S7 or ‘pepL’ targeting the IL-6 receptor alpha subunit, GE11 targeting the EGFR, GRP78p targeting GRP78, pepB1 targeting BMPR1b, pepB2, CLP12, IL-7Ra, GGP, TGFβ-mimic, IL-17Rp, and ACE2p.

In some other illustrative embodiments, this disclosure relates to a composition matter comprising an engineered plasmid vector as disclosed herein, wherein said targeting polypeptide has a sequence of Leu-Ser-Leu-Ile-Thr-Arg-Leu (SEQ ID NO: 1), YHWYGYTPQNVI (SEQ ID NO: 8) targeting the EG, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

In some other illustrative embodiments, this disclosure relates to a composition matter comprising an engineered plasmid vector as disclosed herein, wherein said targeting polypeptide is a combination of a single peptide, homodimers, or heterodimers.

In some other illustrative embodiments, this disclosure relates to a composition matter comprising an engineered plasmid vector as disclosed herein, wherein said optional linker is absent or comprises a single or a plurality of repeated units of Gly-Gly-Gly-Gly-Ser (SEQ ID NO: 2).

In some other illustrative embodiments, this disclosure relates to a composition matter comprising an engineered plasmid vector as disclosed herein, wherein said composition matter further comprising a polymer, wherein said polymer comprises a reverse nuclear localization signal (rNLS), rNLSd, a polycyclooctene polymer with pendant tetralysine and rNLS oligopeptide having a sequence of Val-Lys-Arg-Lys-Lys-Lys-Pro (SEQ ID NO: 4).

In some other illustrative embodiments, this disclosure relates to a method for treating a malignant tumor or an immune disease of a subject comprising the step of administering a therapeutically effective amount of the composition matter as disclosed herein, together with one or more carriers, diluents, or excipients, to the subject in need of relief from said disease.

Yet in some other illustrative embodiments, this disclosure relates to a method for delivery of the gene of a therapeutic protein comprising the steps of
a. preparing an engineered plasmid vector comprising a fusion of a plurality of genes of a therapeutic protein/biologic, a targeting polypeptide, and one or more optional linkers.
b. preparing a polymer comprising a reverse nuclear localization signal (rNLS), called rNLSd, appended onto a polycyclooctene polymer backbone with pendant tetralysine and rNLS oligopeptide having a sequence of Val-Lys-Art-Lys-Lys-Lys-Pro (SEQ ID NO: 4);
c. combining said plasmid vector and said polymer to afform a mixture; and
d. delivering said mixture with an optional aid of sonication (ultrasound-enhanced muscle transfection).

In some illustrative embodiments, this disclosure relates to a method for delivery of the gene of a therapeutic protein according to the steps disclosed herein, wherein said therapeutic protein is a chemokine or a cytokine.

In some illustrative embodiments, this disclosure relates to a method for delivery of the gene of a therapeutic protein according to the steps disclosed herein, wherein said cytokine is selected from the group consisting of interleukin-27 (IL-27) and related cytokines including IL27p28 (IL-30) or EBI3 monomers, IL-23, IL-18, or IL-17 from mouse, human, or canine.

In some illustrative embodiments, this disclosure relates to a method for delivery of the gene of a therapeutic protein according to the steps disclosed herein, wherein said therapeutic protein comprise a sequence of SEQ ID NOs: 5, 6, or 7.

In some illustrative embodiments, this disclosure relates to a method for delivery of the gene of a therapeutic protein according to the steps disclosed herein, wherein said targeting polypeptide further has therapeutic functions.

In some illustrative embodiments, this disclosure relates to a method for delivery of the gene of a therapeutic protein according to the steps disclosed herein, wherein said targeting polypeptide has a sequence of Leu-Ser-Leu-Ile-Thr-Arg-Leu (SEQ ID NO: 1), YHWYGYTPQNVI (SEQ ID NO: 8) targeting the EG, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

In some illustrative embodiments, this disclosure relates to a method for delivery of the gene of a therapeutic protein according to the steps disclosed herein, wherein said optional linker is absent or comprises a single or a plurality of repeated units of Gly-Gly-Gly-Gly-Ser (SEQ ID NO: 3).

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said composition matter comprises
a. an engineered plasmid vector comprising a fusion of a plurality of genes comprising that of a therapeutic protein, a targeting polypeptide, and one or more optional linkers; and
b. a polymer comprising a reverse nuclear localization signal (rNLS), rNLSd, a polycyclooctene polymer with pendant tetralysine and rNLS oligopeptide having a sequence of Val-Lys-Art-Lys-Lys-Lys-Pro (SEQ ID NO: 4).

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said therapeutic protein is a chemokine or a cytokine.

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said cyctokine is selected from the group consisting of interleukin-27 (IL-27) and related cytokines including IL27p28 (IL-30) or EBI3 monomers, IL-23, IL-18, or IL-17 from mouse, human, or canine.

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said therapeutic protein comprise a sequence of SEQ ID NOs: 5, 6, or 7.

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said targeting polypeptide further has therapeutic functions.

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said targeting polypeptide has a sequence of Leu-Ser-Leu-Ile-Thr-Arg-Leu (SEQ ID NO: 1), YHWYGYTPQNVI (SEQ ID NO: 8) targeting the EG, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

Yet in some other illustrative embodiments, this disclosure relates to a method for treating a malignant tumor or an immune disease comprising the step of administering a therapeutically effective amount of a composition matter, together with one or more carriers, diluents, or excipients, to a patient in need of relief, wherein said optional linker is absent or comprises a single or a plurality of repeated units of Gly-Gly-Gly-Gly-Ser (SEQ ID NO: 3).

Below are sets of non-limiting examples to further delineate and explain the invention as disclosed herein.

Engineering of C-term peptide ligands can target Gaussia Luc to tumor cells.

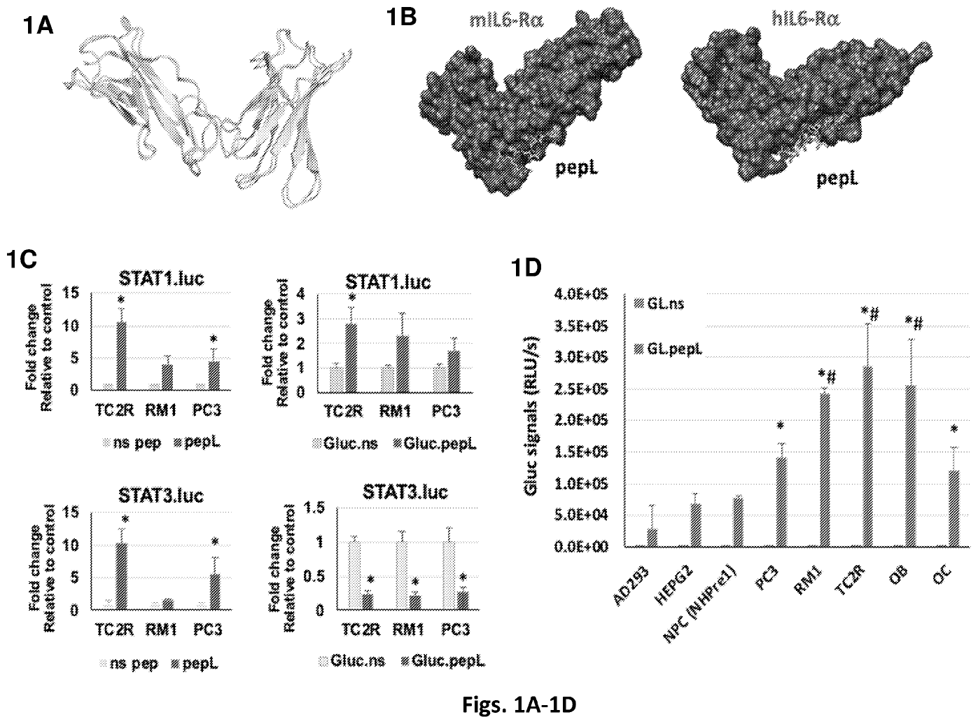

We designed a strategy to target cytokines to the IL-6Ra, a receptor increasingly reported to be upregulated in tumors of various types¹⁵. Because we sought to utilize targeting of cytokines to cells of both human and mouse origin, we generated a model for the mouse IL-6Rα and aligned it to the human IL-6Rα crystal structure model, as described in Materials and Methods. The alignment suggested that the two receptors share high structural homology (FIG. 1a). A peptide previously shown to bind IL-6Rα (pepL, LSLITRL; SEQ ID NO: 1) docks at regions with structural similarity in the receptor models for both species (FIG. 1b). This pepL also has therapeutic activity since it has been reported to reduce signaling through this receptor¹⁵. We also generated a model with the human IL6Rα to confirm that pepL is able to interact with IL6Rα at the interface between IL-6 and the IL-6Ra/gp130 receptor complex.

To model cytokine targeting and detect binding to cells, we designed a Gaussia luciferase (GLuc) molecule modified with the pepL peptide at its C-terminus. We selected Gaussia luciferase as an ideal ‘cytokine model’ since this reporter protein has a signal peptide which enables its secretion from cells. As described in Materials and Methods, Gluc plasmids were engineered to mediate expression of a Gluc protein with a linker and either a control non-specific sequence (Gluc-ns) or the peptide targeting IL6Rα, pepL (Gluc-pepL). In order to best mimic in vivo applications of cytokine-based therapeutics, our rationale was to first produce culture conditioned media (CCM) containing secreted Gluc. The Gluc molecules were expressed by C2C12 muscle cells transfected with a mammalian expression vector, and the CCM was collected for cell binding assays. We utilized firefly luciferase (luc) assays for STAT1 and STAT3 activity to compare the similarities or differences in signaling between the free peptides (ns pep or pepL) with Gluc.ns or Gluc.pepL, where the peptides are linked to the C-terminus of the proteins. The effect of pepL appears to be relatively stronger as a free peptide in terms of STAT1 activation, however, the detrimental effect of activating the oncogenic STAT3 signaling also was observed (FIG. 1c), where STAT3 was upregulated in two prostate cancer cell lines. In contrast, when the peptides are linked to the C-terminus of a protein such as Gluc, the pepL only activated STAT1, whereas STAT3 was significantly downregulated by ˜80% (p<0.05) in three prostate cancer cell lines treated with Gluc-pepL as compared to Gluc-ns control (FIG. 1c). The CCM was used in a binding assay with a variety of human and mouse cells (FIG. id). Normal cells did not bind a significant amount of control (Gluc-ns) or targeted Gluc (Gluc-pepL), as assessed by a Gluc binding assay using CCM in Ad293, HEPG2, or normal prostate epithelial cells (NHPrel), while prostate tumor cells PC3, RM1 and TC2R showed —up to 10-fold increases in Gluc binding relative to Ad293 normal cells. Interestingly, differentiating bone cells (OB, MC3T3E1-14 or OC, RAW264.7) also showed a significant ability to bind Gluc-pepL (FIG. 1d).

Sonoporation delivery in vivo showed that GLuc.pepL can be detected at tumors.

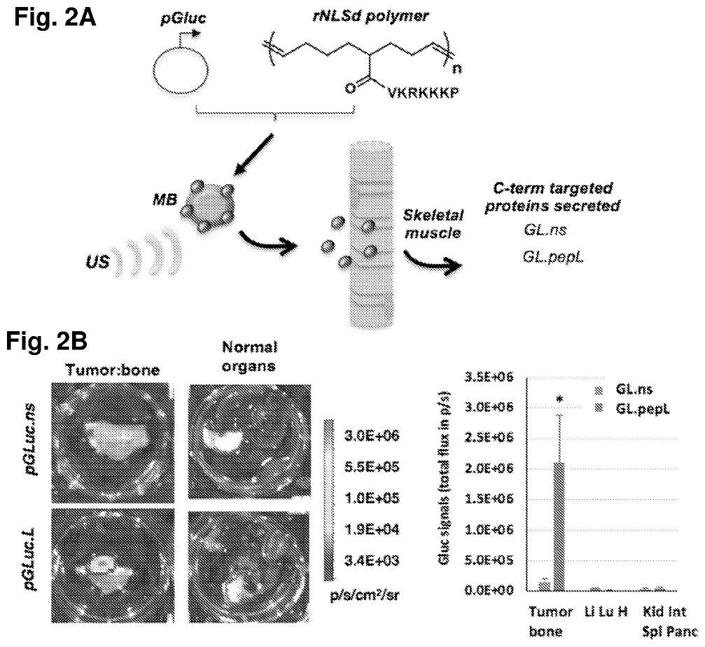

Our group utilizes sonoporation delivery (sonodelivery) to promote protein expression in vivo. FIG. 2a depicts sonodelivery for expressing Gluc proteins in mouse muscle. An ultrasound (US) stimulus is applied to nanoplexes formed by plasmid DNA and cationic polymers in the presence of microbubbles. Following delivery of nanoplex to skeletal muscle, the cytokine model protein (Gluc) is expressed in vivo with a C-terminus peptide/ligand tag (pepL) (FIG. 2a). GLuc is expressed in the hind thigh muscle (dorsally), while the tumor cells are located ventrally, following intratibial implantation (proximal to the knee). Ex vivo evaluation at day 10-14 via sensitive bioluminescence imaging (BLI) showed that signals of targeted GLuc.pepL (but not the control GLuc.ns) were significantly enhanced only in the targeted area (i.e., at the tumor:bone interface), but not in normal organs of mice bearing TC2Ras tumors intratibially (FIG. 2B) (color bar, p/sec/cm²/sr). Normal organs evaluated included the liver, lung, heart, small intestine, kidney, pancreas, and spleen. Remarkably, there was a —13-fold increase in Gluc.pepL accumulation in tumor samples which was significant (p<0.012) relative to untargeted Gluc-treated animals in ex vivo quantification of tumor tissue signals (FIG. 2b, right plot).

We proceeded modify the C-terminus of a cytokine that we previously identified as a promising therapeutic agent for both tumor and bone, IL-27^{3, 4} in the same manner described for Gluc. The mouse EBI3-IL-27p28 ‘hyper IL-27’ was chosen as a fusion protein of the heterodimer components, since it is more potent than delivering each single monomer¹⁷. This IL-27 was then engineered at its C-terminus with a GGGGS linker and peptide ligands pepL or non-specific control (ns) as described in Materials and Methods to generate IL-27pepL or IL-27ns. These C-termini-modified IL-27 vectors were tested in vitro for their ability to express IL-27 (data not shown) and for stimulating IL-27 downstream signaling, as assessed by reporter gene constructs such as STAT1-luc. We reasoned that since free pepL displayed oncogenic STAT3 activation in the reporter assay (FIG. 1c), we proceeded to these next studies solely utilizing the C-terminus linked pepL design, which significantly activated STAT1 while significantly downregulating STAT3 in three prostate cancer cell lines (FIG. 1c). We examined the bioactivity of these C-term-modified IL-27 proteins in vivo as described in the following section.

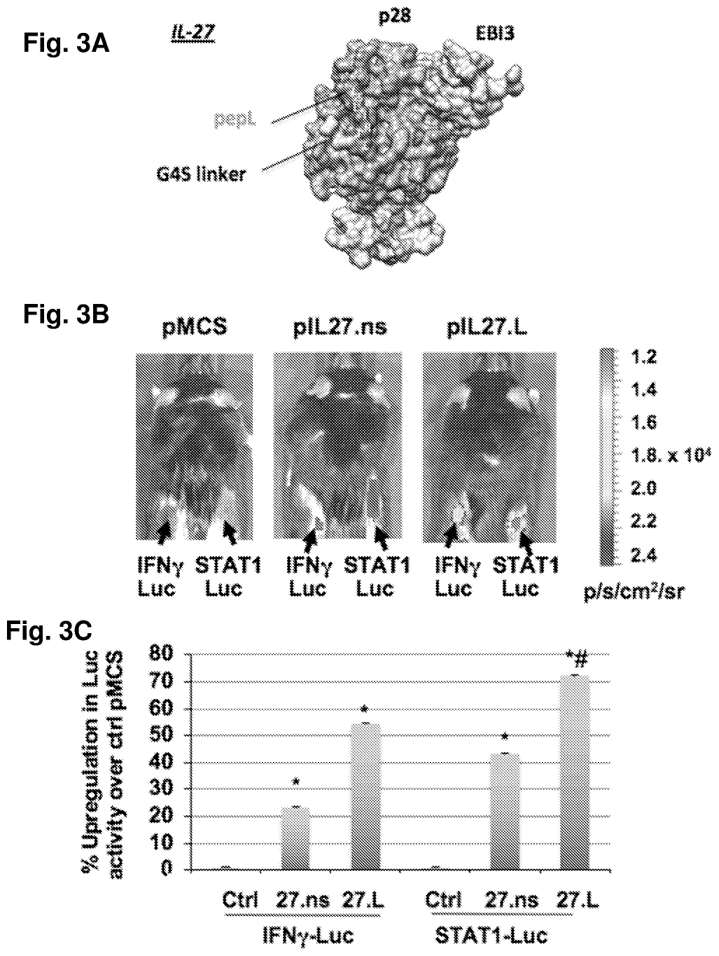

In vivo bioactivity of targeted IL-27pepL is enhanced relative to untargeted IL-27ns. Following generation and examination of a model depicting that pepL would be accessible on the surface of IL-27 (FIG. 3a), we designed an in vivo bioactivity assay whereby implanted “sensor” cells could express reporter gene luciferase in response to IL-27. This assay would enable real-time in vivo detection of IL-27 activity. First, animals received plasmids pMCS (empty vector, pcDNA3.1), pIL-27ns, or pIL-27pepL intramuscularly via sonodelivery to promote cytokine expression (IL-27ns or IL-27pepL) for 3 days. The hind thigh muscle received 12.5 μg of plasmids complexed with polymer rNLSd and microbubbles in the presence of an ultrasound stimulus. Three days after sonodelivery, ‘sensor’ cells (TC2R cells transfected with either STAT1 or IFNγ-responsive Luc vectors) were implanted in the flanks of the animals. TC2R prostate cancer cells were chosen because they exhibit IL6-Rα upregulation. Luciferin substrate was administered intra-peritoneally 24 h later and signals were detected as a surrogate for IL-27 bioactivity (FIG. 3b). STAT1- or IFNγ-luciferase signals were detectable only in animals that received IL-27ns or IL-27pepL (FIG. 3b). Quantification of the bioluminescence signals showed that animals treated with pIL-27ns had a two-fold increase of Luc activity at the cell implantation sites compared to the control vector (pMCS) (FIG. 3c). The animals receiving pIL-27pepL also had a significant increase in luc signals relative to both control pMCS (*, p<0.05) and pIL-27ns (#, p<0.05) (FIG. 3c). This would suggest that a higher bioactivity was achieved by the pepL C-term fusion.

The IL-27 targeting mechanism appears to involve both paracrine and autocrine signaling.

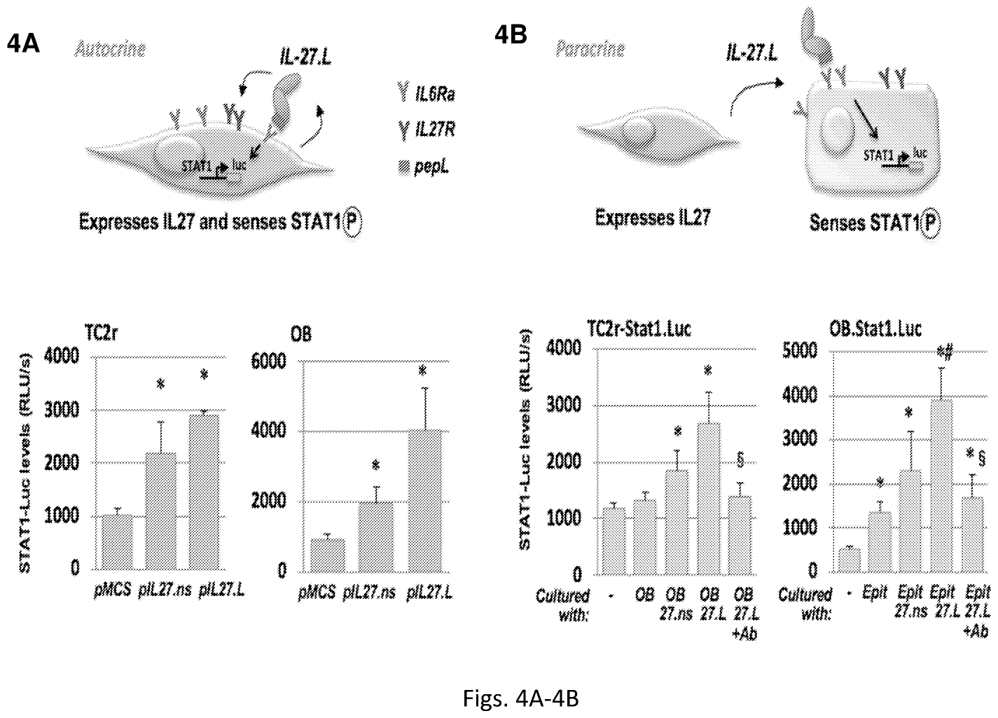

We next examined the potential modes of signaling for the C-term-modified IL-27. We suggest a model by which the peptide allows anchoring of cytokines to cells expressing targeting receptors (for example, IL6Rα) (FIG. 4a). This model proposes that the IL-27 in the CCM could signal in different cells in both Autocrine and Paracrine modes (FIG. 4). We designed an experiment to examine this model, whereby we confirmed that C-term pepL modification enhances IL-27 signaling in vitro. In the autocrine design, pSTAT1-Luc and pIL-27 were co-transfected (FIG. 4a).

In the paracrine design, either differentiating osteoblast (OB, MC3T3E1-14, day 4) or epithelial cells (TC2R) were transfected with STAT1-Luc, then mixed with the other cell type expressing IL-27ns, IL-27pepL or empty vector control (pMCS). In order to signal, IL-27pepL had to be secreted from one cell type and bind to the other cell type (bearing STAT1-luc) to induce signaling (FIG. 4b). In both designs, the C-terminal pepL appeared to enhance IL-27 signaling (p<0.04 vs ctrl, #, p<0.05 vs IL-27) up to 4.4-fold (autocrine design) and up to 3-fold (paracrine design) relative to pMCS or basal co-culture controls. The IL-27pepL-mediated increases in paracrine signaling effect could be blocked by addition of a specific anti-IL-6Rα antibody (FIG. 4b).

IL-27 targeting with pepL modifies gene expression in tumor cells.

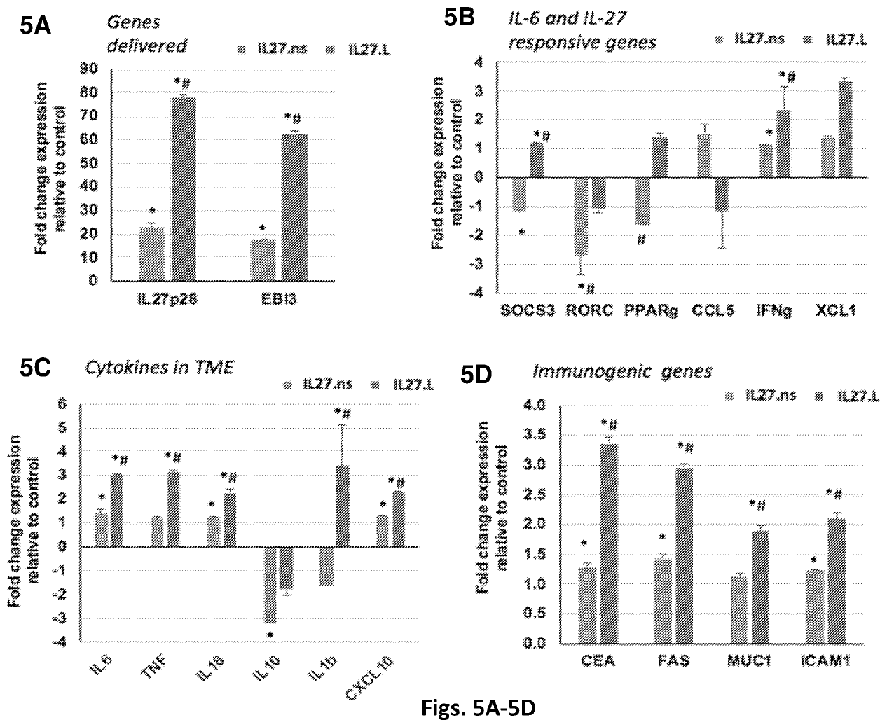

To better understand the potential mechanisms underlying the differences in bioactivity between IL27pepL and IL27ns we examined genes up- or down-regulated by IL-27ns and IL-27pepL relative to control vector (MCS) in transfected TC2R cells. As expected, the IL-27ns vector promoted ˜20-fold upregulation of transgene expression, as assessed by qPCR using primers specific for IL-27p28 and EBI3 subunits (FIG. 5a; p<0.05 relative to control MCS). Interestingly, we observed further upregulation of transgene expression when IL27pepL was delivered, towards a —60-80-fold upregulation of IL-27p28 and EBI3 relative to IL27ns (FIG. 5a; #, p<0.05). The observed upregulation of IL-6 prompted us to query the expression of several target genes associated with IL-6 or IL-27 responses as described in ¹⁸. The IL-27pepL effect differed from the IL-27ns control primarily by promoting significant upregulation of SOCS3 and XCL1 (FIG. 5b). Gene expression of several cytokines relevant to the tumor microenvironment also were assessed, and both IL-27 constructs promoted significant upregulation of IL-6, IL-18, and CXCL10 to ˜2-3-fold (FIG. 5c, *, p<0.05). However, the IL-27pepL construct promoted further upregulation of IL-6, IL-18 and CXCL10, as well as upregulation of TNF and IL1β relative to IL-27ns (FIG. 5c; #, p<0.05). Based on previous studies where IL-27 modulated infiltration of lymphocytes to tumors²′ ⁴, we also examined key immunogenic genes¹⁹. Although all immunogenic genes were significantly upregulated by the IL-27ns relative to control MCS, IL-27pepL delivery significantly enhanced the upregulation by —2-3.5-fold (FIG. 5d). These types of gene expression changes also were confirmed in tumors using qPCR, where we detected significant upregulation of IL27p28, EBI3, TBX21, XCL1, and IFNγ by —2.7-4.9-fold in tumors treated with IL-27pepL relative to IL-27ns (data not shown).

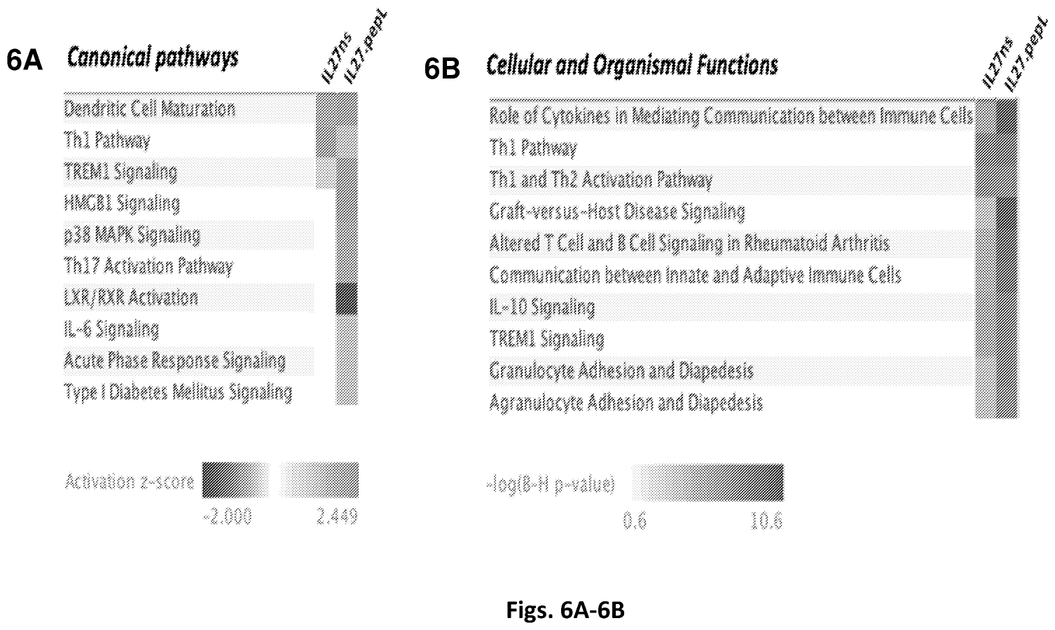

Ingenuity Pathway Analyses (IPA) included (1) Comparison Analyses between TC2R cells treated with IL27ns versus IL27pepL, both corrected for control pMCS qPCR expression levels, and (2) Individual Core Analyses of each treatment group vs. pMCS. Canonical Pathway analyses representations yielded a heatmap with ranked activation z-scores (−2.0 to +2.5) (FIG. 6a) and Cellular and Organismal Functions also ranked in a heatmap by the −log(B-H) of p-values (FIG. 6b), as described in Materials and Methods, and upstream regulators²⁰ (Table 1). Upstream regulator analysis indicated that, relative to control pMCS, the IL27ns-treated TC2R cells had IPA-predicted upstream or causal regulators that included IL-12, LPS-like effect, IFNγ, and TLR4 (p<0.01) and top regulator effect networks that included primarily activation of IL-18, but also FOXO1, IRF4, and IFNγ and inhibition of MYC, collectively relating to the function accumulation of leucocytes. The IL-27pepL-treated TC2R had some of the same IPA-predicted upstream or causal regulators, including IL-12, and TLR4, but some different predicted regulators including IL-27RA, IL-10, and NOD2, relating to the functions lymphoid tissue structure and development and immune cell trafficking. Cellular and organismal functions included communication between immune cells, altered immune cell signaling, IL-10 signaling, and several other immune-related functions.

TABLE 1
qPCR data analyzed by Ingenuity Pathway Analysis - predicted activation
or inhibition and their target molecules in the dataset.

| Treatment | Upstream | | | Upstream | | |
|---|---|---|---|---|---|---|
| relative to | regulators | Fold | | regulators | Fold | |
| control | [symbol] | change | Molecule | [symbol] | change | Molecule |
| pMCS | [symbol] | (>2.0) | type | [symbol] | (<−2.0) | type |
| IL27ns | IL-18 | 2.5 | Cytokine | MYC | −2.2 | Transcription |
| | | | | | | regulator |
| | IFNG | 2.4 | Cytokine | IRF4 | −2.2 | Transcription |
| | | | | | | regulator |
| | RELA | 2.3 | Transcription | NFE2L2 | −2.1 | Transcription |
| | | | regulator | | | regulator |
| | IRF1 | 2.2 | Transcription | IL10 | −2 | Cytokine |
| | | | regulator | | | |
| | FOXO1 | 2.2 | Transcription | | | |
| | | | regulator | | | |
| | TBK1 | 2.2 | Kinase | | | |
| | IL17A | 2.0 | Cytokine | | | |

| Treatment | Upstream | | | Upstream | | |
|---|---|---|---|---|---|---|
| relative to | regulators | Fold | | regulators | Fold | |
| control | [symbol] | change | Molecule | [symbol] | change | Molecule |
| pMCS | [symbol] | (>2.5) | type | [symbol] | (<−2.5) | type |
| IL27pepL | IFNG | 3.3 | Cytokine | SOCS1 | −2.8 | Other |
| | IL12 | 3.2 | Complex | BCL6 | −2.6 | Transcription |
| | | | | | | regulator |
| | IL1B | 3.2 | Cytokine | TNFAIP3 | −2.6 | Enzyme |
| | TNF | 3.1 | Cytokine | IL37 | −2.8 | Cytokine |
| | MYD88 | 3.1 | Other | | | |
| | IL2 | 3.0 | Cytokine | | | |
| | | | Transmembrane | | | |
| | TLR4 | 3.0 | receptor | | | |
| | | | Transmembrane | | | |
| | TLR2 | 3.0 | receptor | | | |
| | | | Transcription | | | |
| | STAT1 | 3.0 | regulator | | | |
| | IL18 | 3.0 | Cytokine | | | |
| | P38MAPK | 3.0 | Group | | | |

^{a}, p-values of overlap, p < 0.001

IL-27 targeting enhances antitumor activity and effector cell recruitment to prostate tumors.

Next we examined the effects of IL-27pepL expression relative to IL-27ns or control (pMCS) vector delivery in vivo. TC2R cells were implanted in C57/BL6 male mice subcutaneously; tumor growth was monitored by caliper measurements. Plasmids (12.5 μg) were delivered to the hind thigh intramuscularly at day 4 using sonoporation. IL-27pepL proved more effective at halting tumor growth than IL-27ns or empty vector control (pMCS) (FIG. 7a; *p<0.05 relative to pMCS control; #, p<0.05 relative to IL-27ns). Tumor growth inhibition was calculated between days 3 and 18, and growth rate was inhibited by 50% for pIL27 and by 89% for pIL27pepL-treated tumors relative to control pMCS-treated tumors. Serum levels of IL-27, detected using ELISA for IL-27p28, showed levels that peaked early on and decreased throughout the study for both IL27ns and IL27pepL (FIG. 7b). Both IL-27-treated groups had significantly higher IL-27 serum levels relative to pMCS control (FIG. 7b) in general, but these increases were only significant for early- and mid-timepoints. The IL27pepL had significantly higher IL27p28 serum levels at the early timepoint relative to IL27ns.

Finally, we examined whether therapy modified extent of tumor-infiltrating lymphocyte populations. We observed a significant upregulation in γδT and NKT for both IL-27 therapies (FIG. 7c; *,p<0.05). However, the IL-27pepL displayed some differences from IL-27ns control, including a higher level of CD3/8 and NKT cells (tp<0.05 relative to IL-27ns), reductions in CD19 cells, and normalization of CD4/25 and NK towards control pMCS-treated levels (p<0.05).

Here we addressed the fusing of C-terminal peptide ligands to Gaussia Luc, a cytokine model protein, and to Interleukin-27, a therapeutic cytokine. We selected a peptide reported to have targeting ability towards the Interleukin-6 receptor alpha (IL-6Ra). Our results suggest that this peptide is effective in vivo to target and treat aggressive prostate tumors, since the receptor and the STAT3 signaling axes are upregulated in —95% of prostate cancer tumor metastases relative to normal tissues²¹. We also observed that this receptor could be useful for targeting differentiating osteoblasts and osteoclasts, and this is supported by the literature, where it has been reported that levels of IL-6Rα are significantly upregulated in vivo as osteoblasts²² and osteoclasts²³ differentiate. The heptapeptide LSLITRL (pepL) was modeled onto the available crystal structure of the hIL6-Ra/gp130 complex, suggesting that the pepL would disrupt signaling through this receptor pair. Also, the mouse/human receptor model alignments, along with our in vitro and in vivo data, indicate that pepL is functional in mouse cells. Signaling through IL6-Rα appeared to be inhibited by the Gluc.pepL fusion but not by free pepL as assessed by STAT3 activity measured using a Luc reporter vector. Also, the effect of pepL appeared to be stronger as a free peptide in terms of STAT1 activation, however, activation of the oncogenic STAT3 signaling also observed suggest that utilizing the free pepL could be detrimental to therapy strategies. This result indicated that the pepL, if provided in the right context (linked at the C-termini), can have a dual targeting and therapeutic function for prostate cancer applications, as has been suggested to have for other tumors¹⁵. Gluc.pepL also could preferentially accumulate at the tumor/bone interface in vivo rather than in normal tissues, implicating this peptide in targeting a cytokine model protein (GLuc) to specific locations. The Gaussia luciferase fusion with pepL (Gluc-pepL) showed a ˜10- to 13-fold increase in binding to tumor cells relative to normal control cells.

Engineering at the C-terminus of the therapeutic cytokine of interest, IL-27, with pepL resulted in higher bioactivity in vivo relative to a non-specific control peptide, as assessed by IFNγ and STAT1 signal detection in responsive cells. This higher bioactivity led us to examine whether the targeting mechanism might involve paracrine and/or autocrine signaling mechanisms. In vitro experiments suggested that the mode of signaling for the IL-27pepL can involve both autocrine and paracrine mechanisms, i.e. it can have effects on the same or neighboring cells and promote STAT1 signaling as assessed by luc reporters. This is important for gene delivery since IL-27 can impact both the targeted cell (tumor) as well as neighboring cells (bone cells or other tumor cells, for example). The experiment shown in FIG. 4 suggests that the chimeric IL27-pepL molecule still can signal through its own receptors since blocking the IL-6Rα with a specific antibody reduced the STAT1 signaling but only to a level equivalent to that of wild-type IL-27. The C-term modified cytokine thus has a dual function (pro-IL27 and anti-IL6 signaling) and constitutes a novel therapeutic cytokine. Overall, the pepL appears to enhance the antitumor activity of IL-27 in vivo, augmenting the protective immune responses that IL-27 already can mount against exogenous and endogenous tumors, which is critical as the basis for future development of an IL-27-based therapeutic agent. The enhanced STAT1 and IFNγ expression utilized in vivo as a surrogate for IL-27's bioactivity were particularly important to validate that a C-term modification (pepL) that enhanced targeting did not disrupt IL-27's ability to signal through these pathways. Combined with the targeting visualized with Gluc-pepL as compared with Gluc-ns, the data suggests that the pepL is able to target cytokines to tumors. When combined with a cytokine such as IL-27, the effect appears to be magnified, enabling further enhancements in IL-27 bioactivity and/or signaling.

Gene expression analyses by qPCR and IPA analyses indicated that the therapeutic cytokines differed in many respects. Interestingly, gene expression results following delivery of control or IL-27 vectors indicated that IL-27pepL potentially has a stronger effect in cells and in vivo. This effect could be attributed to an ability to promote a positive feedback upregulation of IL-27 and regulated genes. Also, IL-27pepL enhances expression of several immunogenic genes and differentially modulates expression of several cytokines that can significantly alter signaling in the tumor microenvironment. Upregulation of TNF, IL-18, IL-1β, and CXCL10 can alter the profile of immune effectors recruited to participate in the immune response against tumors. In particular, CXCL10 has been reported as a chemotactin for NKT and CD8 cells²⁵, and this may underlie the augmented NKT and CD8 infiltration we detected in TC2R tumors. Interestingly, IL-27pepL also upregulated IL-6, perhaps as a compensatory mechanism for the pepL-mediated signaling inhibition. When either IL-6 or IL-27 responsive genes were examined¹⁸, it became apparent that IL-27ns downregulated the three IL-6 responsive genes and upregulated as a trend all three IL-27 responsive genes (although some not significantly). In contrast, IL-27pepL significantly upregulated IL-6 responsive gene SOCS3 and as a trend, PPARy. This activity is likely due to the IL-6 gene expression activation. IL-27pepL significantly upregulated IFNγ and XCL1 (another strong lymphocyte chemotactin), suggesting that the pepL can magnify some while opposing other IL-27 signals. Further development of this IL-27pepL or similarly targeted therapies would aim to reduce IL-6 upregulation and further enhance IL-27 signaling for an augmented therapeutic effect. These types of gene expression changes were confirmed in tumors, where we detected upregulation of IL27p28, EBI3, TBX21, XCL1, and IFNγ when tumors had been treated with IL-27pepL relative to IL-27ns.

Individual IPA analyses of each IL-27 dataset relative to pMCS indicated that several canonical pathways were impacted differently by IL-27pepL relative to IL-27ns. The upstream regulators analysis indicated several potential upstream regulator differences between treatments, and these would be excellent for providing candidates for co-expression to augment efficacy or effect of IL-27pepL therapy in future studies. IPA analyses implicated other networks that can be utilized with IL-27 to potentially achieve synergy in lymphocytic recruitment, including IL-18. Other potential contributing networks that could help balance the IL-6 effects included downregulation of IL-37. IL-37 co-expression along with our vectors could help reduce IL-6 effects by opposing TLR2, 4/Myd88 or p38MAPK-related pro-inflammatory signals. IL-37 is a new IL-1 family member that binds the IL-18 receptor alpha (IL-18Ra) chain, suppresses innate and acquired immunity, and inhibits cytokine levels, including IL-6²⁶. IL-37, IL-18, or IL-12 upregulation could help enhance IL-27 gene delivery protocols, reducing IL-6 or proinflammatory signaling to potentially enhance IL-27 effects. Other regulators upregulated in the IL-27pepL treatment relative to IL-27ns included IFNγ and STAT1, and these might underlie the predicted downregulation of SOCS1²⁷. Reductions in TNFAIP3, a regulator of IRF transcription might underlie the increased IFNγ levels. The gene upregulation showed that IL27pepL upregulates

IL27p28 and EBI3 at higher levels than IL27ns, which could be related to a feed-forward upregulation of STAT1-controlled pathways. STAT1 is a regulator of several IL-27 pathway-related promoter regions²⁸, including EBI3, IL27p28, MYC, RELA, IRF4, IL27RA.

Comparative analyses using IPA of the IL-27 datasets (corrected to baseline pMCS) yielded several interesting canonical pathways and cellular and organismal functions that differed between the two datasets. For example, dendritic cell maturation, TREM1 and HMGB1 signaling were upregulated and LXR/RXR signaling was downregulated. TREM1 signaling could be an underlying cause of the upregulated proinflammatory cytokine genes, while HMGB1 signaling could underlie the upregulation of the immunogenic genes observed. These changes in potential immunity-related processes led us to examine the infiltration of several immune effectors in vivo. The tumor growth inhibition was significant in tumors treated with pIL27 (˜50%) and further enhanced to an 89% growth inhibition in IL-27pepL-treated tumors. This result could be due to several improvements in this therapeutic, including direct effects on the tumor cells (reductions in STAT3), as well as from indirect effects on the tumor such as a higher recruitment of effector cells including a modest but significant increase in CD3/8, a significant decrease in CD19, a normalization of CD4/25, and a significant increase in NKT cells for the IL-27pepL-treated group relative to the mice that received IL27ns gene delivery. Our group and others have shown that for immunogenic tumors, including those of the prostate, IL-27 can inhibit tumor growth and metastasis via increases in CD8 T cells and other effector types^{2, 4, 29}. NKT and CD8 are potent effector lymphocytes with the capacity for killing tumor cells and recruiting other effector cell types; in particular, NKT cells serve as innate immune-regulatory cells. CD19 cell reduction could indicate a loss of B cells in tumors treated with IL-27pepL, as well as normalization of CD4/25 levels compared to IL-27ns, suggesting that IL-27pepL might reverse or normalize to some extent the levels of T_{reg} within tumors. It is interesting that we did not detect increased NK recruitment in this tumor model. The IL-27pepL did not seem to diminish the effect of the cytokine on γδT recruitment, and this is important as γδT cells can recognize and kill tumor cells in a tumor antigen-independent manner, potentially providing protective immune surveillance against metastatic tumors³⁰. Future studies could examine the potential infiltration of other organs by effector cells, although we have not observed any significant lymphocytic infiltration². However, such studies would assess the toxicity potential for this therapeutic modality. In vivo, we observed a significantly higher antitumor activity with the IL27pepL relative to IL27ns. One limitation of our study is that we did not examine the histopathology of the prostate tumors in vivo due to our main focus on the immune effectors infiltrating the tumor, and future studies should incorporate this in the design. Interestingly, when we examine expression levels of IL27, the serum levels were highest for both therapeutics at early timepoints (days 7-11), but declined over time, suggesting a silencing of gene expression. Future studies could employ vectors with hybrid promoters such as hEFla/HTLV or other vectors that could sustain gene expression for longer periods of time.

Additionally, studies combining wild-type or C-term targeted IL-27 with cytokines that modulate different pathways in tumor, bone, and the immune system, including some that are pro-osteogenic, are in progress. Current studies involve strategies to augment the affinity of targeting peptides beyond the micromolar levels of affinity to receptors of interest via homo- or hetero-dimerization. Future studies could explore the ability of the pepL or other related peptides to target cytokines to bone cells and/or bone matrix in vivo to further improve efficacy of IL-27.

Materials and Methods

Cell Culture. Mouse TRAMP—C2 cells were obtained from ATCC and maintained in DMEM:F12 (Mediatech, Manassas, Va.) with 10% FBS and 1× Antibiotic-Antimycotic (AA, Gibco). TRAMP-C2 cells were transduced with a lentivirus expressing activated H-ras^{G12V} at a multiplicity of infection of 1 (m.o.i.=1) plus lentivirus transduction containing the mouse androgen receptor at m.o.i.=1 each to generate the TC2R line², and growth comparisons between the parental TC2 and TC2R were described in ³¹. NHPrel and RM1 were a gift from Dr. S. Hayward. The RM1 murine prostate cancer cell line was described in ². TC2R and RM1 were cultured in DMEM:F12 (Mediatech, Manassas, Va.) with 10% FBS and 1×AA (Gibco). RAW264.7 (murine monocytes) were obtained from ATCC (Manassas, Va., USA) and passaged by utilizing cell lifters. MC-3T3-E1 clone 14 mouse preosteoblasts were obtained from ATCC and cultured in 10% heat inactivated ATCC FBS in alpha-MEM (Invitrogen) media with 1×AA (Gibco). HepG2, AML12, HEK293, and C2C12 were obtained from ATCC and grown in DMEM with 10% FBS and 1×AA (Gibco). Normal prostate cells (Rwpel or NHprel) were either obtained from ATCC or as a generous gift from S. Hayward and grown using Keratinocyte Serum Free Medium kit (ATCC). PC3 were obtained from ATCC and grown in RPMI1640 with 10% FBS and 1×AA (Gibco). All cells except for RAW264.7 were passaged by trypsinization (0.05% (v/v) trypsin, 0.53 mM EDTA) (Gibco).

Culture Conditioned Media and Differentiation. For the Gluc binding or STATS reporter assays, conditioned culture media (CCM) was obtained from C2C12 muscle cells as follows: C2C12 cells were grown to 70-80% confluence, transfected using Lipofectamine 2000 with plasmids, media changed after 6 h to complete DMEM/10% FBS and cells allowed to recover overnight (˜16 h). The next day, cells were washed 2× in PBS, and received 2% DMEM:F12/1×AA (Gibco) and CCM collected 48 h later. Input CCM used in the GLuc binding assay did not display significant differences in luminescence levels (data not shown). For differentiating MC3T3E1 clone 14 cells into osteoblasts, heat-inactivation of FBS (ATCC) was carried out at 55° C. for 30 min, followed by storage at 4° C. prior to addition to media. Differentiating osteoblasts (OB) were obtained by treating MC3T3E1 for 1 week with ascorbic acid and beta-glycerol phosphate from an osteogenesis kit (Millipore, ECM810) prior to GLuc cell binding assays. For differentiating RAW264.7 mouse cells into osteoclasts, cells were cultured in DMEM/10% FBS with 1×AA and gently scraped for passaging. These cells were differentiated into osteoclasts (OC) by 35 ng/ml RANKL (RnD systems) treatment in complete media for 6 days prior to cell binding assays.

Peptide, Receptor Analysis Techniques, and Luciferase Assays. For firefly luciferase (Luc) reporter assays, constructs responsive to the active (phosphorylated) form of STAT1 were used (STAT1.GAS/ISRE-Luc; LR0026, Panomics, Fremont, Calif.) or IFN□-Luc (Addgene, #17599) to transfect cells using Lipofectamine 2000 according to the manufacturer's protocols for each cell type and cytokine stimulation as described in ³². For the STAT3-luc assay, C2C12 CCM was generated as described above, then CCM incubated with HEK293, PC3, RM1, or TC2R cells which had been transfected with STAT3-luc vector (Signosis, LR-2004 Panomics, Fremont, Calif.) using Lipofectamine 2000. Free peptides were synthesized and obtained from Selleckchem (Houston, Tex.). Cells were collected at 5 h or 24 h of IL-27 (or control) stimulation, lysed in passive lysis buffer (Promega, Madison, Wis.) and assayed in 96-well format using a Glomax luminometer with luciferin substrate (Promega).

For the paracrine versus autocrine mode of action for IL-27 experiment, using STAT1-luc assays, pepL-modified IL-27 was compared to pIL27ns or empty pcDNA3.1 control vector (pMCS) via assessing whether modified IL-27 signals in Autocrine and Paracrine modes. In the paracrine design, either differentiating osteoblast (OB, MC3T3E1-14) or TC2r epithelial cells were transfected with STAT1-Luc using lipofectamine 2000. The next day, cells were lifted, counted, and then 3×10³ of each cell type was co-mixed with the other cell type expressing IL-27ns, IL-27pepL or empty vector ctrl in 2% FBS media in 96-well white plates. In order to signal, IL-27pepL had to be secreted from one cell type and bind to the other cell type (bearing STAT1-luc) to induce signaling. In the autocrine design, pSTAT1-Luc and pIL-27s were cotransfected in the same cell. An antibody used to block IL-6Rα signaling was added at cell seeding in the paracrine experiment at 0.2 ug in 100 uL (Biolegend, 115811).

For Gluc binding assays, CCM was generated as described above and utilized to treat cells seeded (10⁴/well for OB, 6×10⁴/well OC, and 3×10⁴/well for others) in a 96-well format in a white plate (Corning), and levels of Gluc in the input were equivalent across samples (data not shown). CCM was allowed to incubate with cells at 37C 5%CO₂ for 16h, media removed, washed with 1×DPBS, and cells lysed in 1× Renilla lysis buffer (Promega) 40uL. 50-100 uL Renilla substrate was added and plate was read using a Glomax luminometer (Promega) with 10 sec integration time. Results are displayed as RLU/sec.

For analyses of pepL binding to the IL6-Rα chain, we utilized the human IL6-Rα PDB 1p9m file to model the interactions. The alignment of human and mouse IL6-Rα was done using PyMol following modeling of the mouse IL6-Rα by iTASSER³³. PepL was docked to both human and mouse IL6-Rα utilizing GalaxyPepDock³⁴, which enables prediction of 3D protein-peptide complex structure interactions from input protein structure and peptide sequence information using similar interactions found in the structure database and energy-based optimization. The modeling predicted a similar location in the IL6Rα structure for binding of pepL, supporting the interaction with this receptor in both species.

Vectors. Plasmid DNA vectors for IL-27 expression were prepared using a pcDNA3.1 backbone. PCR cloning was utilized to clone the hyper-IL-27 cDNA from pORF9-mEBI3/p28 (Invivogen) with a 3′ insertion of a sequence encoding peptide linker (GGGGS; SEQ ID NO: 2)³⁵ plus the targeting peptide sequences (s7 or pepL: LSLITRL; SEQ ID NO: 1 and as a non-specific (ns) control: EDLGREK (SEQ ID NO: 3), previously shown to lack any specificity for IL6/gp130³⁶). IL-27 cDNA-linker-peptide sequences were subcloned into pDrive (Promega), then excised and cloned into pcDNA3.1 using BamHI and Nhel ends; empty vector control was pcDNA3.1-MCS (pMCS). Vectors were prepared for all experiments using Endofree kits (Qiagen, Valencia, Calif.). For efficient complexation with polymer, vectors were first precipitated and resuspended in water. Briefly, precipitation used 1:10 volume 3M NaOAc and 2 volumes of cold 100% ethanol, followed by a 30 min incubation at −80° C. and centrifugation at 12,000 rpm for 15 min at 4° C., and a wash using 2 volumes of 70% ethanol with a 5 min spin at room temp. The pellet was allowed to dry and was resuspended in sterile nuclease free water. Sonoporation of vectors intramuscularly has been described in detail previously¹³.

Ingenuity pathway analyses (IPA) and real time PCR. For qPCR, we performed transfection of TC2R cells in a 6-well format using 5×10⁵ cells and Lipofectamine 3000 according to manufacturer's protocols (Invitrogen), to introduce pcDNA3.1 empty vector (pMCS), or expressing IL27ns or IL27pepL, and collected RNA at 24 h post-transfection. The cDNA synthesis and qPCR followed procedures previously published by our group³, with mouse-specific primers (sequences available upon request). For network analyses, upstream regulator analysis, and downstream effect analysis, real time qPCR data were inputted into Ingenuity Pathway Analysis (IPA, QIAGEN Redwood City) as described in ³⁷. qPCR data were generated using gene-specific primers, as described in ³. Briefly, by comparing the imported qPCR data with the Ingenuity Knowledge Base, a list of relevant networks, upstream regulators and algorithmically generated mechanistic networks based on their connectivity was obtained. Only genes with a p-value ≤0.05 were considered and both direct and indirect relationships were considered. Upstream regulator analysis was used to predict the upstream transcriptional regulators from the dataset based on the literature and compiled in the Ingenuity Knowledge Base. The analysis examines how many known targets of the upstream regulators are present in treated cell datasets and also the direction of change as compared to control. An overlap p-value is computed based on significant overlap between genes in the dataset and known targets regulated by the transcriptional regulator, with an activation z-score algorithm to make predictions. Downstream effect analysis was used to predict activation state (increased or decreased) if the direction of change is consistent with the activation state of a biological function. Top functions (cell and organismal functions) were scored by IPA and plotted as a heatmap with p value <2.2e-12 and sorted by predicted activation and by number of molecules, and the top 10 pathways or cellular/organismal functions were depicted. IPA calculates a Benjamini-Hochberg (B-H) corrected p-value for Upstream Regulators and for Causal Networks, increasing the statistical stringency of these results in Core Analyses.

In vivo studies and intratumoral lymphocyte infiltration by FACS. Animal care and procedures were performed in accordance with the Purdue University institutional review board guidelines (PACUC). For bioactivity assays in vivo, TC2R cells were transfected with luciferase reporter vectors containing either STAT1 binding sites or the IFNγ promoter to generate ‘reporter cells’. Equal numbers of reporter cells (7.7×10⁵) were implanted in the flanks of C57BL6 males (n=6) that had received in the hind thigh 2 days prior by sonoporation 12.5 μg of plasmid DNA (either empty control pMCS, IL-27 with a non-specific peptide (ns) at the C-terminus, or C-term-targeted IL-27 (IL-27pepL)). pDNA were delivered via sonodelivery (polymer NLSd+ultrasound+MB). After reporter cell injection, animals were imaged for Luc activity at day 3 or day 7 post-sonoporation of pDNA. Bioluminescent signals were detectable using an IVIS100 Xenogen imager only in animals that received pIL-27ns or pIL-27pepL but not pMCS control vector. For tumor implantation for IL-27 therapeutic studies, we trypsinized TC2R cells grown in in DMEM:F12 with 10% FBS and 1×AA, washed in 1×DBPS centrifugation step, then re-suspended the pellet in sterile 1×DPBS and kept the cells on ice prior to implantation under isoflurane anesthesia. Male C57/BL6 mice (8-10 weeks of age) flanks were shaved and 5×10⁵ TC2R cells implanted subcutaneously. Tumor growth was monitored over time using Vernier calipers to generate tumor volume measurements in mm³.

For gene delivery, we utilized the polymer containing a reverse nuclear localization signal (rNLS), rNLSd, a polycyclooctene polymer with pendant tetralysine and rNLS oligopeptide (VKRKKKP; SEQ ID NO: 4), synthesized as described in the literatures^{13, 38}. We prepared polymers in low retention Eppendorf tubes, dissolved in nuclease-free water, and sterilized by filtration. The stock solution of NLSd was diluted to enable complexation with pGluc plasmid DNA at an N/P ratio of 6 (i.e., the ratio of protonatable nitrogens in the polymer, N, to DNA phosphates, P). DNA (12.5 μg) in nuclease-free water was combine with polymer in nuclease-free water at a 1:1 ratio and allowed to equilibrate for a minimum of 35 min under sterile conditions. Following polyplex formation, 5.5% sterile Micromarker microbubbles (VisualSonics, Toronto, Ontario, Canada) were added per tube and injected intramuscularly to the hind legs of male mice. After applying ultrasound gel, we sonoporated the muscle to mediate gene delivery of GLuc or IL-27 plasmids using a Sonigene instrument (VisualSonics) with 1 MHz, 20% duty cycle, and 3 W/cm² for 60 sec. In vivo imaging for luciferase expression in muscle was performed starting on day 4 following sonoporation using previously published procedures by intravenous luciferin substrate administration and collection of images within 15 min using an IVIS Imager with a CCD apparatus^{39,40}. For the IL-27 therapy study, we administered plasmids once intramuscularly on day 4 (tumor sizes in average of ˜30mm³). The mice were randomized by tumor size in 3 groups relative to treatment tested, with n=6 per group (pMCS, pIL27ns, pIL27pepL). Flow cytometry for infiltrating lymphocyte detection utilized methods and antibodies previously described⁴.

Statistical analyses. Assays were performed in triplicate and values provided as mean±SEM or 95% confidence interval. Comparisons were performed using unpaired t-tests or one-way analysis of variance analysis using the Bonferroni t-test and p<0.05 considered to indicate a significant difference.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Figueiredo Neto, M, and Figueiredo, M L (2017). Combination of Interleukin-27 and MicroRNA for Enhancing Expression of Anti-Inflammatory and Proosteogenic Genes. Arthritis 2017: 6365857.
2. Zolochevska, O, Xia, X., Williams, B J, Ramsay, A., Li, S., Figueiredo, M. L. (2011). Sonoporation delivery of Interleukin 27 gene therapy efficiently reduces prostate tumor cell growth in vivo. Human Gene Therapy.
3. Zolochevska, O, Diaz-Quiñones, A O, Ellis, J, and Figueiredo, M L (2013). Interleukin-27 expression modifies prostate cancer cell crosstalk with bone and immune cells in vitro. J Cell Physiol 228: 1127-1136.
4. Zolochevska, O, Ellis, J, Parelkar, S, Chan-Seng, D, Emrick, T, Wei, J, et al. (2013). Interleukin-27 gene delivery for modifying malignant interactions between prostate tumor and bone. Hum Gene Ther 24: 970-981.
5. Figueiredo, M L (2017). Editorial: IL-27 expression following TLR activation in bone: sounding the alarm for repair. J Leukoc Biol 101: 1276-1279.
6. Dibra, D, Cutrera, J J, Xia, X, Birkenbach, M P, and Li, S (2009). Expression of WSX1 in tumors sensitizes IL-27 signaling-independent natural killer cell surveillance. Cancer Res 69: 5505-5513.
7. Larousserie, F, Bsiri, L, Dumaine, V, Dietrich, C, Audebourg, A, Radenen-Bussière, B, et al. (2017). Frontline Science: Human bone cells as a source of IL-27 under inflammatory conditions: role of TLRs and cytokines. J Leukoc Biol 101: 1289-1300.
8. Yoshimoto, T, Morishima, N, Mizoguchi, I, Shimizu, M, Nagai, H, Oniki, S, et al. (2008). Antiproliferative activity of IL-27 on melanoma. J Immunol 180: 6527-6535.
9. Pradhan, A, Lambert, Q T, and Reuther, G W (2007). Transformation of hematopoietic cells and activation of JAK2-V617F by IL-27R, a component of a heterodimeric type I cytokine receptor. Proc Natl Acad Sci U S A 104: 18502-18507.
10. Lucas, S, Ghilardi, N, Li, J, and de Sauvage, F J (2003). IL-27 regulates IL-12 responsiveness of naive CD4+T cells through Statl-dependent and -independent mechanisms. Proc Natl Acad Sci USA 100: 15047-15052.
11. Takeda, A, Hamano, S, Yamanaka, A, Hanada, T, Ishibashi, T, Mak, T W, et al. (2003). Cutting edge: role of IL-27/WSX-1 signaling for induction of T-bet through activation of STAT1 during initial Thl commitment. J Immunol 170: 4886-4890.
12. Zhu, S, Lee, D A, and Li, S (2010). IL-12 and IL-27 sequential gene therapy via intramuscular electroporation delivery for eliminating distal aggressive tumors. J Immunol 184: 2348-2354.
13. Parelkar, S S, Letteri, R, Chan-Seng, D, Zolochevska, O, Ellis, J, Figueiredo, M, et al. (2014). Polymer-peptide delivery platforms: effect of oligopeptide orientation on polymer-based DNA delivery. Biomacromolecules 15: 1328-1336.
14. Meka, R R, Venkatesha, S H, and Moudgil, K D (2018). Peptide-directed liposomal delivery improves the therapeutic index of an immunomodulatory cytokine in controlling autoimmune arthritis. J Control Release 286: 279-288.
15. Su, J L, Lai, K P, Chen, C A, Yang, C Y, Chen, P S, Chang, C C, et al. (2005). A novel peptide specifically binding to interleukin-6 receptor (gp80) inhibits angiogenesis and tumor growth. Cancer Res 65: 4827-4835.
16. Ellis, J, Falzon, M, Emrick, T, and Figueiredo, M L (2014). Imaging cytokine targeting to the tumor/bone microenvironment in vivo. Hum Gene Ther Clin Dev 25: 200-201.
17. Rousseau, F, Basset, L, Froger, J, Dinguirard, N, Chevalier, S, and Gascan, H (2010). IL-27 structural analysis demonstrates similarities with ciliary neurotrophic factor (CNTF) and leads to the identification of antagonistic variants. Proc Natl Acad Sci U S A 107: 19420-19425.
18. Hirahara, K, Onodera, A, Villarino, A V, Bonelli, M, Sciumé, G, Laurence, A, et al. (2015). Asymmetric Action of STAT Transcription Factors Drives Transcriptional Outputs and Cytokine Specificity. Immunity 42: 877-889.
19. Ardiani, A, Farsaci, B, Rogers, C J, Protter, A, Guo, Z, King, T H, et al. (2013). Combination therapy with a second-generation androgen receptor antagonist and a metastasis vaccine improves survival in a spontaneous prostate cancer model. Clin Cancer Res 19: 6205-6218.
20. Krämer, A, Green, J, Pollard, J, and Tugendreich, S (2014). Causal analysis approaches in Ingenuity Pathway Analysis. Bioinformatics 30: 523-530.
21. Don-Doncow, N, Marginean, F, Coleman, I, Nelson, P S, Ehrnstrom, R, Krzyzanowska, A, et al. (2017). Expression of STAT3 in Prostate Cancer Metastases. Eur Urol 71: 313-316.
22. Li, Y, Bäckesjö, C M, Haldosén, L A, and Lindgren, U (2008). IL-6 receptor expression and IL-6 effects change during osteoblast differentiation. Cytokine 43: 165-173.
23. Zhao, H, Zhang, X, Chen, X, Li, Y, Ke, Z, Tang, T, et al. (2014). Isoliquiritigenin, a flavonoid from licorice, blocks M2 macrophage polarization in colitis-associated tumorigenesis through downregulating PGE2 and IL-6. Toxicol Appl Pharmacol 279: 311-321.
24. Natividad, K D, Junankar, S R, Mohd Redzwan, N, Nair, R, Wirasinha, R C, King, C, et al. (2013). Interleukin-27 signaling promotes immunity against endogenously arising murine tumors. PLoS One 8: e57469.
25. Wei, J, Xia, S, Sun, H, Zhang, S, Wang, J, Zhao, H, et al. (2013). Critical role of dendritic cell-derived IL-27 in antitumor immunity through regulating the recruitment and activation of NK and NKT cells. J Immunol 191: 500-508.
26. Conti, P, Ronconi, G, Kritas, S K, Caraffa, A, and Theoharides, TC (2018). Activated Mast Cells Mediate Low-Grade Inflammation in Type 2 Diabetes: Interleukin-37 Could Be Beneficial. Can J Diabetes 42: 568-573.
27. Sandling, J K, Gamier, S, Sigurdsson, S, Wang, C, Nordmark, G, Gunnarsson, I, et al. (2011). A candidate gene study of the type I interferon pathway implicates IKBKE and IL8 as risk loci for SLE. Eur J Hum Genet 19: 479-484.
28. Rouillard, A D, Gundersen, G W, Fernandez, N F, Wang, Z, Monteiro, C D, McDermott, M G, et al. (2016). The harmonizome: a collection of processed datasets gathered to serve and mine knowledge about genes and proteins. Database (Oxford) 2016.
29. Murugaiyan, G, and Saha, B (2013). IL-27 in tumor immunity and immunotherapy. Trends Mol Med 19: 108-116.
30. Dieli, F, Vermijlen, D, Fulfaro, F, Caccamo, N, Meraviglia, S, Cicero, G, et al. (2007). Targeting human {gamma}delta} T cells with zoledronate and interleukin-2 for immunotherapy of hormone-refractory prostate cancer. Cancer Res 67: 7450-7457.
31. Umbaugh, CS, Diaz-Quiñones, A, Neto, M F, Shearer, J J, and Figueiredo, M L (2018). A dock derived compound against laminin receptor (37 LR) exhibits anti-cancer properties in a prostate cancer cell line model. Oncotarget 9: 5958-5978.
32. Bluyssen, H A, and Levy, D E (1997). Stat2 is a transcriptional activator that requires sequence-specific contacts provided by statl and p48 for stable interaction with DNA. J Biol Chem 272: 4600-4605.
33. Roy, A, Kucukural, A, and Zhang, Y (2010). I-TASSER: a unified platform for automated protein structure and function prediction. Nat Protoc 5: 725-738.
34. Lee, H, Heo, L, Lee, M S, and Seok, C (2015). GalaxyPepDock: a protein-peptide docking tool based on interaction similarity and energy optimization. Nucleic Acids Res 43: W431-435.
35. Sockolosky, J T, Kivimäe, S, and Szoka, F C (2014). Fusion of a short peptide that binds immunoglobulin G to a recombinant protein substantially increases its plasma half-life in mice. PLoS One 9: e102566.
36. Manfredini, R, Tenedini, E, Siena, M, Tagliafico, E, Montanari, M, Grande, A, et al. (2003). Development of an IL-6 antagonist peptide that induces apoptosis in 7TD1 cells. Peptides 24: 1207-1220.
37. Gopurappilly, R, and Bhonde, R (2015). Transcriptional profiling and functional network analyses of islet-like clusters (ILCs) generated from pancreatic stem cells in vitro. Genomics 105: 211-219.
38. Parelkar, S S, Chan-Seng, D, and Emrick, T (2011). Reconfiguring polylysine architectures for controlling polyplex binding and non-viral transfection. Biomaterials 32: 2432-2444.
39. Zolochevska, O, and Figueiredo, M L (2009). Expression of cell cycle regulator cdk2apl suppresses tumor cell phenotype by non-cell-autonomous mechanisms. Oral Oncol 45: e106-112.
40. Zolochevska, O, Yu, G, Gimble, J M, and Figueiredo, M L (2012). Pigment epithelial-derived factor and melanoma differentiation associated gene-7 cytokine gene therapies delivered by adipose-derived stromal/mesenchymal stem cells are effective in reducing prostate cancer cell growth. Stem Cells Dev 21: 1112-1123.

1. A composition of matter comprising an engineered plasmid vector, wherein said vector comprises a fusion of a plurality of genes of a therapeutic chemokine or a cytokine, a targeting polypeptide, and one or more optional linkers.

2. The composition of matter of claim 1, wherein said cytokine is selected from the group consisting of interleukin-27 (IL-27), IL27p28 (IL-30), Epstein-Barr virus-induced gene 3 (EBI3), IL-23, IL-18, IL-17, and any combination thereof.

3. The composition of matter of claim 2, wherein said cytokine is origin of a mouse, a human, or a canine.

4. The composition of matter of claim 2, wherein said cytokine is a IL-27 comprised of linked subunits of IL27B (EBI3) and IL27A (IL27p28) having a sequence of:
MSKLLFLSLALWASRSPGYTETALVALSQPRVQCH
ASRYPVAVDCSWTPLQAPNSTRSTSFIATYRLGVA
TQQQSQPCLQRSPQASRCTIPDVHLFSTVPYMLNV
TAVHPGGASSSLLAFVAERIIKPDPPEGVRLRTAG
QRLQVLWHPPASWPFPDIFSLKYRLRYRRRGASHF
RQVGPIEATTFTLRNSKPHAKYCIQVSAQDLTDYG
KPSDWSLPGQVESAPHKPVPGVGVPGVGFPTDPLS
LQELRREFTVSLYLARKLLSEVQGYVHSFAESRLP
GVNLDLLPLGYHLPNVSLTFQAWHHLSDSERLCFL
ATTLRPFPAMLGGLGTQGTWTSSEREQLWAMRLDL
RDLHRHLRFQVLAAGFKCSKEEEDKEEEEEEEEEE
KKLPLGALGGPNQVSSQVSWPQLLYTYQLLHSLEL
VLSRAVRDLLLLSLPRRPGSAWDS
(SEQ ID NO: 5; mouse IL27 with
linked subunits of IL27B (EBI3)
and IL27A (IL27p28));
or
MTPQLLLALVLWASCPPCSGRKGPPAALTLPRVQC
RASRYPIAVDCSWTLPPAPNSTSPVSFIATYRLGM
AARGHSWPCLQQTPTSTSCTITDVQLFSMAPYVLN
VTAVHPWGSSSSFVPFITEHIIKPDPPEGVRLSPL
AERQLQVQWEPPGSWPFPEIFSLKYWIRYKRQGAA
RFHRVGPIEATSFILRAVRPRARYYIQVAAQDLTD
YGELSDWSLPATATMSLGKVPGVGVPGVGFPRPPG
RPQLSLQELRREFTVSLHLARKLLAEVRGQAHRFA
ESHLPGVNLYLLPLGEQLPDVSLTFQAWRRLSDPE
RLCFISTTLQPFHALLGGLGTQGRWTNMERMQLWA
MRLDLRDLQRHLRFQVLAAGFNLPEEEEEEEEEEE
EERKGLLPGALGSALQGPAQVSWPQLLSTYRLLHS
LELVLSRAVRELLLLSKAGHSVWPLGFPTLSPQP
(SEQ ID NO: 6; human IL27 linked subunits IL27B (EBI3) and IL27A (IL27p28)) ; or
MAPGLLLVLALWVGCSPCRGREGAPAAPTQPRVRC
RASRYPVAVDCFWTLPPAPRSATPTSFIATYRLGV
AAHGESLPCLQQTPEATSCTIPDVHMFSMVPYVLN
VTAVRPWGSSSSFVPFVPEQLIKPDPPEGVRLSVL
PRQRLWVQWEPPRSWPFPELFSLKYWIRYKHHGSP
RFRQVGPIEATSFTFRAVRPQARYCIQVAAQDLTD
YGESSDWSLPAAPSTPLGKVPGVGVPGVGFPRPPG
RSPLSLQELRREFKVSLQLAKKLFSEVRIQAHHFA
ESQLPGVSLDLLPLGDQLPNVSLPFQAWHSLSDPE
RLCFLSMMLHPFHALLESLGSQGGWTSSEKMHLWT
MRLDLRDLQRHLRFQVEYPPTCSTPRDQQEEEEEQ
HEERKGLLAAAPGGPSQTAVQPSWPQLLYTYQLLH
SLELALARAVRDLLLLSQAGNPAPPVGHSTFGSQP
(SEQ ID NO: 7; CANINE IL27 with linked subunits of IL27B (EBI3) and IL27A (IL27p28)).

5. Canceled.

6. The composition of matter of claim 1, wherein said targeting polypeptide comprises S7 or ‘pepL’ targeting the IL-6 receptor alpha subunit, GE11 targeting the EGFR, GRP78p targeting GRP78, pepB1 targeting BMPR1b, pepB2, CLP12, IL-7Ra, GGP, TGFβ-mimic, IL-17Rp, and ACE2p.

7. The composition of matter of claim 6, wherein said targeting polypeptide has a sequence of Leu-Ser-Leu-Ile-Thr-Arg-Leu (SEQ ID NO: 1), YHWYGYTPQNVI (SEQ ID NO: 8) targeting the EGFR, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

8. (canceled)

9. The composition of matter of claim 1, wherein said optional linker is absent or comprises a single or a plurality of repeated units of Gly-Gly-Gly-Gly-Ser (SEQ ID NO: 2).

10. The composition of matter of claim h further comprising a polymer, wherein said polymer comprises a reverse nuclear localization signal (rNLS), rNLSd, a polycyclooctene polymer with pendant tetralysine and a rNLS oligopeptide having a sequence of Val-Lys-Arg-Lys-Lys-Lys-Pro (SEQ ID NO: 4).

11. A method for treating a malignant tumor or an immune disease of a subject comprising the step of administering a therapeutically effective amount of the composition of matter of claim 1, together with one or more carriers, diluents, or excipients, to the subject.

12. A method for delivery of the gene of a therapeutic protein comprising the steps of:
a. preparing an engineered plasmid vector comprising a fusion of a plurality of genes of a therapeutic protein/biologic, a targeting polypeptide, and one or more optional linkers;
b. preparing a polymer comprising a reverse nuclear localization signal (rNLS), called rNLSd, appended onto a polycyclooctene polymer backbone with pendant tetralysine and rNLS oligopeptide having a sequence of Val-Lys-Art-Lys-Lys-Lys-Pro (SEQ ID NO: 4);
c. combining said plasmid vector and said polymer to afford a mixture; and
d. delivering said mixture with an optional aid of sonication (ultrasound-enhanced muscle transfection).

13. The method of claim 12, wherein said therapeutic protein is a chemokine or a cytokine.

14. The method of claim 13, wherein said cytokine is selected from the group consisting of interleukin-27 (IL-27) and related cytokines including IL27p28 (IL-30) or EBI3 monomers, IL-23, IL-18, or IL-17 from mouse, human, or canine.

15. The method of claim 12, wherein said therapeutic protein comprises a sequence of SEQ ID NOs: 5, 6, or 7.

16. Canceled.

17. The method of claim 12, wherein said targeting polypeptide has a sequence of Leu-Ser-Leu-Ile-Thr-Arg-Leu (SEQ ID NO: 1), YHWYGYTPQNVI (SEQ ID NO: 8) targeting the EG, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

18. The method of claim 12, wherein said optional linker is absent or comprises a single or a plurality of repeated units of Gly-Gly-Gly-Gly-Ser (SEQ ID NO: 3).

19. A method for treating a malignant tumor or an immune disease comprising the step of administering a therapeutically effective amount of a composition of matter, together with one or more carriers, diluents, or excipients, to a patient in need of relief, wherein said composition of matter comprises:
a. an engineered plasmid vector comprising a fusion of a plurality of genes comprising that of a therapeutic protein, a targeting polypeptide, and one or more optional linkers; and
b. a polymer comprising a reverse nuclear localization signal (rNLS), rNLSd, a polycyclooctene polymer with pendant tetralysine and rNLS oligopeptide having a sequence of Val-Lys-Art-Lys-Lys-Lys-Pro (SEQ ID NO: 4).

20. The method of claim 19, wherein said therapeutic protein is a chemokine or a cytokine.

21. The method of claim 20, wherein said cytokine is selected from the group consisting of interleukin-27 (IL-27) and related cytokines including IL27p28 (IL-30) or EBI3 monomers, IL-23, IL-18, or IL-17 from mouse, human, or canine.

22. The method of claim 19, wherein said therapeutic protein comprises a sequence of SEQ ID NOs: 5, 6, or 7.

23. (canceled)

24. The method of claim 19, wherein said targeting polypeptide has a sequence of Leu-Ser-Leu-Ile-Thr-Arg-Leu (SEQ ID NO: 1), YHWYGYTPQNVI (SEQ ID NO: 8) targeting the EG, SNTRVAP (SEQ ID NO: 9) targeting GRP78, AISMLYLDENEKVVL (SEQ ID NO: 10) targeting BMPR1b, TPLSYLKGLVTV (SEQ ID NO: 11), NPYHPTIPQSVH (SEQ ID NO: 12), ASACPPH (SEQ ID NO: 13), GGPNLTGRW (SEQ ID NO: 14), FLPASGL (SEQ ID NO: 15, TGFβ-mimic), TPIVHHVA (SEQ ID NO: 16), or TVALPGGYVRV (SEQ ID NO: 17).

25. The method of claim 19, wherein said optional linker is absent or comprises a single or a plurality of repeated units of Gly-Gly-Gly-Gly-Ser (SEQ ID NO: 3).
